(19) United States
(12) Patent Application Publication
(10) Pub. No.: US 2025/0109112 A1
(43) Pub. Date: Apr. 3, 2025

(54) HERBICIDAL COMPOUNDS

(71) Applicant: SYNGENTA CROP PROTECTION AG, Basel (CH)

(72) Inventor: William Guy WHITTINGHAM, Bracknell, Berkshire (GB)

(73) Assignee: SYNGENTA CROP PROTECTION AG, Basel (CH)

(21) Appl. No.: 18/833,155

(22) PCT Filed: Jan. 19, 2023
(86) PCT No.: PCT/EP2023/051243

§ 371 Date: Jul. 25, 2024

(30) Foreign Application Priority Data
Jan. 26, 2022 (EP) 22153395.3

(51) Int. Cl. C07D 251/38; A01N 43/66; A01P 13/00

(52) U.S. Cl. CPC C07D 251/38; A01N 43/66; A01P 13/00

(57) ABSTRACT

Compound of formula (I) or an agronomically acceptable salt thereof: (I) wherein the substituents are as defined in claim 1, useful as a pesticides, especially as herbicides.

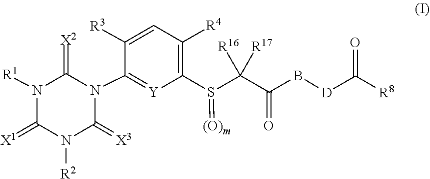

The present invention relates to herbicidally active triazine derivatives, as well as to processes and intermediates used for the preparation of such derivatives. The invention further extends to herbicidal compositions comprising such derivatives, as well as to the use of such compounds and compositions for controlling undesirable plant growth: in particular the use for controlling weeds, in crops of useful plants.

WO2019/121543, WO2021/018664, and WO2021/259224 all disclose herbicidal compounds similar to those of the present invention.

The present invention is based on the finding that triazine derivatives of formula (I) as defined herein, exhibit surprisingly good herbicidal activity. Thus, according to the present invention there is provided a compound of formula (I) or an agronomically acceptable salt thereof:

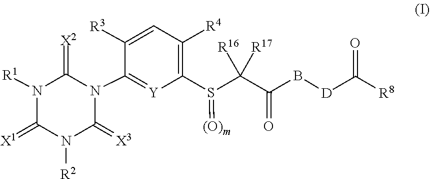

wherein
each X¹, X² and X³ is independently selected from oxygen and sulfur;
Y is C—H or nitrogen;
B is O, S, or NR⁵;
D is (CR⁶R⁷)ₙ;
m is an integer from 0 to 2;
n is an integer from 1 to 4;
R¹ is hydrogen or C₁-C₆alkyl;
R² is hydrogen, amino, C₁-C₆alkyl, C₃-C₆alkenyl, or C₃-C₆alkynyl;
R³ is hydrogen, halogen, C₁-C₄alkyl, C₁-C₄haloalkyl, C₁-C₄alkoxy, C₁-C₄haloalkoxy, C₁-C₄alkylthio, or C₁-C₄alkylsulfonyl;
R⁴ is hydrogen, halogen, cyano, nitro, aminocarbonyl, aminothiocarbonyl, C₁-C₄alkyl, C₁-C₄haloalkyl, C₁-C₄alkoxy, C₁-C₄haloalkoxy, or C₁-C₄alkylsulfonyl;
R⁵ is hydrogen, hydroxy, C₁-C₆alkyl, or C₁-C₄alkoxy;
Each R⁶ and R⁷ is independently selected from hydrogen, halogen, C₁-C₆alkyl, C₁-C₆haloalkyl, hydroxy, C₁-C₄alkoxy, C₁-C₄alkoxycarbonyl, or CH₂OR¹²; provided that R⁶ and R⁷ are not both hydroxy on the same carbon atom;
or two groups R⁶ and R⁷, on the same or different carbon atoms, together form a C₁-C₅alkylene chain, which contain 0, 1 or 2 oxygen atoms, substituted by 1-3 groups R¹⁵;
or two groups R⁶ and R⁷, on the same carbon atom, together with the carbon to which they are attached, form a C₂alkene;
R⁸ is OR⁹, SR⁹, or NR¹⁰R¹¹;
R⁹ is hydrogen, C₁-C₁₀alkyl, C₁-C₁₀haloalkyl, C₃-C₆alkenyl, C₃-C₆haloalkenyl, C₃-C₆alkynyl, C₁-C₄alkoxyC₁-C₆alkyl, C₁-C₄haloalkoxyC₁-C₆alkyl, C₆-C₁₀arylC₁-C₃alkyl, C₆-C₁₀arylC₁-C₃alkyl substituted by 1-4 groups R¹³, heteroarylC₁-C₃alkyl, or heteroarylC₁-C₃alkyl substituted by 1-3 groups R¹³;
R¹⁰ is hydrogen, C₁-C₆alkyl, or SO₂R¹⁴;
R¹¹ is hydrogen or C₁-C₆alkyl; or
R¹⁰ and R¹¹ together with the nitrogen to which they are attached form a 3- to 6-membered heterocyclyl ring, which optionally contains an oxygen atom;
R¹² is hydrogen, C₁-C₄alkyl, or C₁-C₄alkylcarbonyl;
each R¹³ is independently selected from halogen, C₁-C₄alkyl, C₁-C₄haloalkyl, C₁-C₄alkoxy, C₁-C₄haloalkoxy, cyano, and C₁-C₄alkylsulfonyl;
R¹⁴ is C₁-C₄alkyl, C₁-C₄haloalkyl, or C₁-C₄alkyl(C₁-C₄alkyl)amino; each R¹⁵ is independently selected from hydrogen, halogen, C₁-C₄alkyl, and C₁-C₄haloalkyl;
R¹⁶ and R¹⁷ are independently selected from hydrogen, halogen, C₁-C₆alkyl, C₁-C₆haloalkyl, C₁-C₄alkoxy, and CH₂OR¹²;
or two groups R¹⁶ and R¹⁷, together form a C₂-C₅alkylene chain, which contains 0, 1 or 2 oxygen atoms, substituted by 1-3 groups R¹⁵;
or two groups R¹⁶ and R¹⁷ together with the carbon to which they are attached form a C₂alkene;
or a salt or N-oxide thereof.

According to a second aspect of the invention, there is provided an agrochemical composition comprising a herbicidally effective amount of a compound of formula (I) and an agrochemically-acceptable diluent or carrier. Such an agricultural composition may further comprise at least one additional active ingredient.

According to a third aspect of the invention, there is provided a method of controlling or preventing undesirable plant growth, wherein a herbicidally effective amount of a compound of formula (I), or a composition comprising this compound as active ingredient, is applied to the plants, to parts thereof or the locus thereof.

According to a fourth aspect of the invention, there is provided the use of a compound of formula (I) as a herbicide.

According to a fifth aspect of the invention, there is provided a process for the preparation of compounds of formula (I).

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, hydroxy means an —OH group.

As used herein, nitro means an —NO₂ group.

As used herein, amino means an —NH₂ group.

As used herein, the term “C₁-C₁₀alkyl” refers to a straight or branched hydrocarbon chain radical consisting solely of carbon and hydrogen atoms, containing no unsaturation, having from one to ten carbon atoms, and which is attached to the rest of the molecule by a single bond. C₁-C₆alkyl, C₁-C₄alkyl and C₁-C₂alkyl are to be construed accordingly. Examples of C₁-C₁₀alkyl include, but are not limited to, methyl (Me), ethyl (Et), n-propyl, 1-methylethyl (iso-propyl), n-butyl, and 1-dimethylethyl (t-butyl).

As used herein, the term “C₁-C₄alkoxy” refers to a radical of the formula —ORₐ where Rₐ is a C₁-C₄alkyl radical as generally defined above. C₁-C₃alkoxy is to be construed accordingly. Examples of C₁₋₄alkoxy include, but are not limited to, methoxy, ethoxy, propoxy, iso-propoxy and t-butoxy.

As used herein, the term “C₁-C₁₀haloalkyl” refers to a C₁-C₁₀alkyl radical as generally defined above substituted by one or more of the same or different halogen atoms. C₁-C₆haloalkyl and C₁-C₄haloalkyl are to be construed accordingly. Examples of C₁-C₁₀haloalkyl include, but are not limited to chloromethyl, fluoromethyl, fluoroethyl, difluoromethyl, trifluoromethyl and 2,2,2-trifluoroethyl.

As used herein, the term “C₂-C₆alkenyl” refers to a straight or branched hydrocarbon chain radical group consisting solely of carbon and hydrogen atoms, containing at least one double bond that can be of either the (E)- or (2)-configuration, having from two to six carbon atoms, which is attached to the rest of the molecule by a single bond. C₃-C₆alkenyl and C₂-C₄alkenyl are to be construed accordingly. Examples of C₂-C₆alkenyl include, but are not limited to, prop-1-enyl, allyl (prop-2-enyl) and but-1-enyl.

As used herein, the term “C₂-C₆haloalkenyl” refers to a C₂-C₆alkenyl radical as generally defined above substituted by one or more of the same or different halogen atoms. C₃-C₆haloalkenyl and C₂-C₄haloalkenyl are to be construed accordingly. Examples of C₂-C₆haloalkenyl include, but are not limited to chloroethylene, fluoroethylene, 1,1-difluoroethylene, 1,1-dichloroethylene and 1,1,2-trichloroethylene.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. C₃-C₆alkynyl and C₂-C₄alkynyl are to be construed accordingly. Examples of C₂-C₆alkynyl include, but are not limited to, prop-1-ynyl, propargyl (prop-2-ynyl) and but-1-ynyl.

As used herein, the term “C₁-C₄haloalkoxy” refers to a C₁-C₄alkoxy group as defined above substituted by one or more of the same or different halogen atoms. C₁-C₃haloalkoxy is to be construed accordingly. Examples of C₁-C₄haloalkoxy include, but are not limited to, fluoromethoxy, difluoromethoxy, fluoroethoxy, trifluoromethoxy and trifluoroethoxy.

As used herein, the term “C₁-C₄haloalkoxyC₁-C₆alkyl” refers to a radical of the formula R_{b}—O—Rₐ— where R_{b} is a C₁-C₄haloalkyl radical as generally defined above, and Rₐ is a C₁-C₆alkylene radical as generally defined above.

As used herein, the term “C₁-C₄alkoxyC₁-C₆alkyl” refers to a radical of the formula R_{b}—O—Rₐ— where R_{b} is a C₁-C₄alkyl radical as generally defined above, and Rₐ is a C₁-C₆alkylene radical as generally defined above.

As used herein, the term “C₁-C₄alkylcarbonyl” refers to a radical of the formula —C(O)Rₐ where Rₐ is a C₁-C₄alkyl radical as generally defined above. C₁-C₂alkylcarbonyl is to be construed accordingly.

As used herein, the term “C₁-C₄alkoxycarbonyl” refers to a radical of the formula —C(O)ORₐ where Rₐ is a C₁-C₄alkyl radical as generally defined above.

As used herein, the term “aminocarbonyl” refers to a radical of the formula —C(O)NH₂.

As used herein, the term “aminothiocarbonyl” refers to a radical of the formula —C(S)NH₂. As used herein, the term “C₁-C₄alkylthio” refers to a radical of the formula —SRₐ, where Rₐ is a C₁-C₄alkyl radical as generally defined above.

As used herein, the term “C₁-C₄alkylsulfonyl” refers to a radical of the formula —S(O)₂Rₐ, where Rₐ is a C₁-C₄alkyl radical as generally defined above. The terms “C₁-C₃alkylsulfonyl” and “C₁-C₂alkylsulfonyl”, are to be construed accordingly. Examples of C₁-C₆alkylsulfonyl include, but are not limited to methylsulfonyl.

As used herein, the term “C₆-C₁₀aryl” refers to a 6- to 10-membered aromatic ring system consisting solely of carbon and hydrogen atoms which may be mono-, bi- or tricyclic. Examples of such ring systems include phenyl, naphthalenyl, or indenyl.

As used herein, the term “C₆-C₁₀arylC₁-C₃alkyl” refers to an aryl moiety as generally defined above, which is attached to the rest of the molecule by a C₁-C₃alkylene linker as defined above. As used herein, the term “C₁-C₄alkyl(C₁-C₄alkyl)amino” refers to a radical of the formula Rₐ(R_{b})NH—, wherein Rₐ and R_{b} are both C₁-C₄alkyl radicals as generally defined above.

As used herein, except where explicitly stated otherwise, the term “heteroaryl” refers to a 5- or 6-membered monocyclic aromatic ring which comprises 1, 2, 3 or 4 heteroatoms individually selected from nitrogen, oxygen and sulfur. The heteroaryl radical may be bonded to the rest of the molecule via a carbon atom or heteroatom. Examples of heteroaryl include, furyl, pyrrolyl, imidazolyl, thienyl, pyrazolyl, thiazolyl, isothiazolyl, oxazolyl, isoxazolyl, triazolyl, tetrazolyl, pyrazinyl, pyridazinyl, pyrimidyl or pyridyl.

As used herein, except where explicitly stated otherwise, the term “heterocyclyl” or “heterocyclic” refers to a stable 4- to 6-membered non-aromatic monocyclic ring radical which comprises 1, 2, or 3 heteroatoms individually selected from nitrogen, oxygen and sulfur. The heterocyclyl radical may be bonded to the rest of the molecule via a carbon atom or heteroatom. Examples of heterocyclyl include, but are not limited to, pyrrolinyl, pyrrolidyl, tetrahydrofuryl, tetrahydrothienyl, tetrahydrothiopyranyl, piperidyl, piperazinyl, tetrahydropyranyl, dihydroisoxazolyl, dioxolanyl, morpholinyl or δ-lactamyl.

The presence of one or more possible asymmetric carbon atoms in a compound of formula (I) means that the compounds may occur in chiral isomeric forms, i.e., enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers (including lactam-lactim tautomerism and keto-enol tautomerism) where present. The present invention includes all possible tautomeric forms for a compound of formula (I). Similarly, where there are di-substituted alkenes, these may be present in E or Z form or as mixtures of both in any proportion. The present invention includes all these possible isomeric forms and mixtures thereof for a compound of formula (I).

The compounds of formula (I) will typically be provided in the form of an agronomically acceptable salt, a zwitterion or an agronomically acceptable salt of a zwitterion. This invention covers all such agronomically acceptable salts, zwitterions and mixtures thereof in all proportions.

Suitable agronomically acceptable salts of the present invention can be with cations that include but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium and tripropylsulfoxonium.

The following list provides definitions, including preferred definitions, for substituents X¹, X², X³, Y, B, D, m, n, R¹, R², R³, R⁴, R⁵, R⁶, R⁷, R⁸, R⁹, R¹⁰, R¹¹, R¹², R¹³, R¹⁴, R¹⁵, R¹⁶ and R¹⁷ with reference to the compounds of formula (I) according to the invention. For any one of these substituents, any of the definitions given below may be combined with any definition of any other substituent given below or elsewhere in this document.
X¹ is oxygen or sulfur. Preferably, X¹ is sulfur.
X² is oxygen or sulfur. Preferably, X² is oxygen.
X³ is oxygen or sulfur. Preferably, X³ is oxygen.
Y is C—H or nitrogen. Preferably, Y is C—H.
B is O, S or NR⁵. Preferably, B is O, NH, or NMe. More preferably, B is O or NH.
m is an integer from 0 to 2. Preferably, m is 0 or 2, more preferably, m is 0.
n is an integer from 1 to 4. Preferably, n is an integer from 1 to 2, more preferably, n is 2.
R¹ is hydrogen or C₁-C₆alkyl. Preferably, R¹ is hydrogen or C₁-C₄alkyl. More preferably, R¹ is C₁-C₂alkyl, and most preferably, R¹ is methyl.
R² is hydrogen, amino, C₁-C₆alkyl, C₃-C₆alkenyl or C₃-C₆alkynyl. Preferably, R² is hydrogen, C₁-C₄alkyl, or C₃-C₄alkynyl. More preferably R² is C₁-C₂alkyl, and most preferably R² is methyl.
R³ is hydrogen, halogen, C₁-C₄alkyl, C₁-C₄haloalkyl, C₁-C₄alkoxy, C₁-C₄haloalkoxy, C₁-C₄alkylthio, or C₁-C₄alkylsulfonyl. Preferably, R³ is hydrogen, chloro, or fluoro. More preferably, R³ is hydrogen or fluoro. More preferably still, R³ is fluoro.
R⁴ is hydrogen, halogen, cyano, nitro, aminocarbonyl, aminothiocarbonyl, C₁-C₄alkyl, C₁-C₄haloalkyl, C₁-C₄alkoxy, C₁-C₄haloalkoxy, or C₁-C₄alkylsulfonyl. Preferably, R⁴ is hydrogen, chloro, bromo, cyano, or aminothiocarbonyl. More preferably, R⁴ is chloro, bromo, or cyano, and most preferably R⁴ is chloro.
R⁵ is hydrogen, hydroxy, C₁-C₆alkyl, or C₁-C₄alkoxy. Preferably, R⁵ is hydrogen or C₁-C₄alkyl. More preferably, R⁵ is hydrogen or C₁-C₂alkyl. Even more preferably, R⁵ is hydrogen or methyl. In one set of embodiments, R⁵ is hydrogen.
Each R⁶ and R⁷ is independently selected from hydrogen, halogen, C₁-C₆alkyl, C₁-C₆haloalkyl, hydroxy, C₁-C₄alkoxy, C₁-C₄alkoxycarbonyl, and CH₂OR¹²; provided that R⁶ and R⁷ are not both hydroxy on the same carbon atom;
or two groups R⁶ and R⁷, on the same or different carbon atoms, together form a C₁-C₅alkylene chain, which contain 0, 1 or 2 oxygen atoms, substituted by 1-3 groups R¹⁵;
or two groups R⁶ and R⁷, on the same carbon atom, together with the carbon to which they are attached, form a C₂alkene. Preferably, each R⁶ and R⁷ is independently selected from hydrogen, halogen, C₁-C₄alkyl, and C₁-C₄alkoxycarbonyl. More preferably, each R⁶ and R⁷ is independently selected from hydrogen, halogen, and C₁-C₂alkyl. Most preferably, each R⁶ and R⁷ is independently selected from hydrogen, chloro, and methyl. In one embodiment, R⁶ and R⁷ are both hydrogen.
R⁸ is OR⁹, SR⁹, or NR¹⁰R¹¹. Preferably, R⁸ is OR⁹. In one embodiment, R⁸ is methoxy.
R⁹ is hydrogen, C₁-C₁₀alkyl, C₁-C₁₀haloalkyl, C₃-C₆alkenyl, C₃-C₆haloalkenyl, C₃-C₆alkynyl, C₁-C₄alkoxyC₁-C₆alkyl, C₁-C₄haloalkoxyC₁-C₆alkyl, C₆-C₁₀arylC₁-C₃alkyl, C₆-C₁₀arylC₁-C₃alkyl substituted by 1-4 groups R¹³, heteroarylC₁-C₃alkyl, or heteroarylC₁-C₃alkyl substituted by 1-3 groups R¹³.

Preferably, R⁹ is selected from hydrogen, C₁-C₄alkyl, C₁-C₄haloalkyl, C₁-C₂alkoxyC₁-C₂alkyl, phenylC₁-C₂alkyl and phenylC₁-C₂alkyl substituted by 1-2 groups R¹³. More preferably, R⁹ is hydrogen, C₁-C₄alkyl, C₁-C₂alkoxyC₁-C₂alkyl, or phenylC₁-C₂alkyl. More preferably still, R⁹ is hydrogen, C₁-C₄alkyl, or phenylC₁-C₂alkyl. Even more preferably, R⁹ is C₁-C₃alkyl. In a particularly preferred embodiment, R⁹ is methyl.
R¹⁰ is hydrogen, C₁-C₆alkyl, or SO₂R¹⁴. Preferably, R¹⁰ is hydrogen or SO₂R¹⁴. More preferably R¹⁰ is SO₂R¹⁴.
R¹¹ is hydrogen or C₁-C₆alkyl. Preferably, R¹¹ is hydrogen.
R¹² is hydrogen, C₁-C₄alkyl, or C₁-C₄alkylcarbonyl. Preferably, R¹² is hydrogen, C₁-C₂alkyl, or C₁-C₂alkylcarbonyl. More preferably, R¹² is hydrogen or methyl.
Each R¹³ is independently selected from halogen, C₁-C₄alkyl, C₁-C₄haloalkyl, C₁-C₄alkoxy, C₁-C₄haloalkoxy, cyano, and C₁-C₄alkylsulfonyl. Preferably, R¹³ is selected from halogen, C₁-C₄alkyl, C₁-C₄haloalkyl, C₁-C₄alkoxy, C₁-C₄haloalkoxy, cyano, and C₁-C₄alkylsulfonyl.
R¹⁴ is C₁-C₄alkyl, C₁-C₄haloalkyl, or C₁-C₄alkyl(C₁-C₄alkyl)amino. Preferably, R¹⁴ is C₁-C₄alkyl or C₁-C₄alkyl(C₁-C₄alkyl)amino. More preferably, R¹⁴ is methyl or isopropyl(methyl)amino.
Each R¹⁵ is independently selected from hydrogen, halogen, C₁-C₄alkyl, and C₁-C₄haloalkyl. Preferably, each R¹⁵ is independently selected hydrogen, halogen, and C₁-C₂alkyl. More preferably, each R¹⁵ is independently selected from hydrogen and methyl. Most preferably, R¹⁵ is hydrogen.
R¹⁶ and R¹⁷ are independently selected from hydrogen, halogen, C₁-C₆alkyl, C₁-C₆haloalkyl, C₁-C₄alkoxy, and CH₂OR¹². Preferably, R¹⁶ and R¹⁷ are independently selected from hydrogen, C₁-C₄alkyl and C₁-C₂alkoxy. More preferably, R¹⁶ and R¹⁷ are independently selected from hydrogen and C₁-C₂alkyl. Most preferably, R¹⁶ and R¹⁷ are independently selected from hydrogen and methyl.

A preferred subset of compounds is one in which;
X¹ is sulfur;
X² is oxygen or sulfur;
X³ is oxygen;
Y is C—H;
B is O or NH;
m is 0;
n is 1 or 2;
R¹ is C₁-C₂alkyl;
R² is C₁-C₂alkyl;
R³ is selected from hydrogen, chloro, and fluoro;
R⁴ is selected from chloro, bromo, and cyano;
each R⁶ and R⁷ is independently selected from hydrogen, halogen and C₁-C₂alkyl;
R⁸ is OR⁹;
R⁹ is selected from hydrogen, C₁-C₄alkyl, C₁-C₂alkoxyC₁-C₂alkyl and phenylC₁-C₂alkyl;
R¹⁶ and R¹⁷ are independently selected from hydrogen and C₁-C₂alkyl.

A more preferred subset of compounds is one in which;
X¹ is sulfur;
X² is oxygen;
X³ is oxygen;
B is NH;
m is 0;
n is 2;
Y is C—H;
R¹ is methyl;
R² is methyl;
R³ is selected from hydrogen and fluoro:
R⁴ is chloro;
each R⁶ and R⁷ is independently selected from hydrogen and methyl;
R⁸ is OR⁹;
R⁹ is selected from hydrogen, C₁-C₄alkyl and phenylC₁-C₂alkyl;
R¹⁶ and R¹⁷ are independently selected from hydrogen and methyl.

A second more preferred subset of compounds is one in which;
X¹ is sulfur;
X² is oxygen;
X³ is oxygen;
B is 0;
m is 0;
n is 2;
Y is C—H;
R¹ is methyl;
R² is methyl;
R³ is selected from hydrogen and fluoro;
R⁴ is chloro;
each R⁶ and R⁷ is independently selected from hydrogen and methyl;
R⁸ is OR⁹;
R⁹ is selected from hydrogen, C₁-C₄alkyl and phenylC₁-C₂alkyl;
R¹⁶ and R¹⁷ are independently selected from hydrogen and methyl.

In one set of preferred embodiments, X¹ is sulfur;
X² is oxygen;
X³ is oxygen;
B is O or NH;
m is 0;
n is 2;
Y is C—H;
R¹ is methyl;
R² is methyl;
R³ is fluoro;
R⁴ is chloro;
R⁶ and R⁷ are both hydrogen;
R⁸ is methoxy;
R¹⁶ and R¹⁷ are each independently selected from hydrogen and methyl.

### Tables of Examples

Table 1 below discloses 1230 specific compounds of formula (I), designated compounds 1-1 to 1-1230 respectively, wherein X¹ is sulfur, X² and X³ are oxygen, R¹ and R² are methyl, R⁴ is chloro, R¹⁶ and R¹⁷ are hydrogen, and Y is C-H.

| Compound | | | | | |
|---|---|---|---|---|---|
| Number | m | R³ | B | D | R⁸ |
| 1-1 | 0 | H | O | CH₂ | OH |
| 1-2 | 0 | H | O | CH₂ | OMe |
| 1-3 | 0 | H | O | CH₂ | OEt |
| 1-4 | 0 | H | O | CH₂ | OCH₂Ph |
| 1-5 | 0 | H | O | CH₂ | NH₂ |
| 1-6 | 0 | H | O | CH₂ | NHEt |
| 1-7 | 0 | H | O | CH₂ | NHSO₂Me |
| 1-8 | 0 | H | O | CH₂ | NHSO₂NMe₂ |
| 1-9 | 0 | H | O | CH₂ | NHSO₂N(Me)(CHMe₂) |
| 1-10 | 0 | F | O | CH₂ | OH |
| 1-11 | 0 | F | O | CH₂ | OMe |
| 1-12 | 0 | F | O | CH₂ | OEt |
| 1-13 | 0 | F | O | CH₂ | OCH₂Ph |
| 1-14 | 0 | F | O | CH₂ | NH₂ |
| 1-15 | 0 | F | O | CH₂ | NHEt |
| 1-16 | 0 | F | O | CH₂ | NHSO₂Me |
| 1-17 | 0 | F | O | CH₂ | NHSO₂NMe₂ |
| 1-18 | 0 | F | O | CH₂ | NHSO₂N(Me)(CHMe₂) |
| 1-19 | 0 | Cl | O | CH₂ | OH |
| 1-20 | 0 | Cl | O | CH₂ | OMe |
| 1-21 | 0 | Cl | O | CH₂ | OEt |
| 1-22 | 0 | Cl | O | CH₂ | OCH₂Ph |
| 1-23 | 0 | Cl | O | CH₂ | NH₂ |
| 1-24 | 0 | Cl | O | CH₂ | NHEt |
| 1-25 | 0 | Cl | O | CH₂ | NHSO₂Me |
| 1-26 | 0 | Cl | O | CH₂ | NHSO₂NMe₂ |
| 1-27 | 0 | Cl | O | CH₂ | NHSO₂N(Me)(CHMe₂) |
| 1-28 | 0 | H | NH | CH₂ | OH |
| 1-29 | 0 | H | NH | CH₂ | OMe |
| 1-30 | 0 | H | NH | CH₂ | OEt |
| 1-31 | 0 | H | NH | CH₂ | OCH₂Ph |
| 1-32 | 0 | H | NH | CH₂ | NH₂ |
| 1-33 | 0 | H | NH | CH₂ | NHEt |
| 1-34 | 0 | H | NH | CH₂ | NHSO₂Me |
| 1-35 | 0 | H | NH | CH₂ | NHSO₂NMe₂ |
| 1-36 | 0 | H | NH | CH₂ | NHSO₂N(Me)(CHMe₂) |
| 1-37 | 0 | F | NH | CH₂ | OH |
| 1-38 | 0 | F | NH | CH₂ | OMe |
| 1-39 | 0 | F | NH | CH₂ | OEt |
| 1-40 | 0 | F | NH | CH₂ | OCH₂Ph |
| 1-41 | 0 | F | NH | CH₂ | NH₂ |
| 1-42 | 0 | F | NH | CH₂ | NHEt |
| 1-43 | 0 | F | NH | CH₂ | NHSO₂Me |
| 1-44 | 0 | F | NH | CH₂ | NHSO₂NMe₂ |
| 1-45 | 0 | F | NH | CH₂ | NHSO₂N(Me)(CHMe₂) |
| 1-46 | 0 | Cl | NH | CH₂ | OH |
| 1-47 | 0 | Cl | NH | CH₂ | OMe |
| 1-48 | 0 | Cl | NH | CH₂ | OEt |
| 1-49 | 0 | Cl | NH | CH₂ | OCH₂Ph |
| 1-50 | 0 | Cl | NH | CH₂ | NH₂ |
| 1-51 | 0 | Cl | NH | CH₂ | NHEt |
| 1-52 | 0 | Cl | NH | CH₂ | NHSO₂Me |
| 1-53 | 0 | Cl | NH | CH₂ | NHSO₂NMe₂ |
| 1-54 | 0 | Cl | NH | CH₂ | NHSO₂N(Me)(CHMe₂) |
| 1-55 | 0 | H | O | CHMe | OH |
| 1-56 | 0 | H | O | CHMe | OMe |
| 1-57 | 0 | H | O | CHMe | OEt |
| 1-58 | 0 | H | O | CHMe | OCH₂Ph |
| 1-59 | 0 | H | O | CHMe | NH₂ |
| 1-60 | 0 | H | O | CHMe | NHEt |
| 1-61 | 0 | H | O | CHMe | NHSO₂Me |
| 1-62 | 0 | H | O | CHMe | NHSO₂NMe₂ |
| 1-63 | 0 | H | O | CHMe | NHSO₂N(Me)(CHMe₂) |
| 1-64 | 0 | F | O | CHMe | OH |
| 1-65 | 0 | F | O | CHMe | OMe |
| 1-66 | 0 | F | O | CHMe | OEt |
| 1-67 | 0 | F | O | CHMe | OCH₂Ph |
| 1-68 | 0 | F | O | CHMe | NH₂ |
| 1-69 | 0 | F | O | CHMe | NHEt |
| 1-70 | 0 | F | O | CHMe | NHSO₂Me |
| 1-71 | 0 | F | O | CHMe | NHSO₂NMe₂ |
| 1-72 | 0 | F | O | CHMe | NHSO₂N(Me)(CHMe₂) |
| 1-73 | 0 | Cl | O | CHMe | OH |
| 1-74 | 0 | Cl | O | CHMe | OMe |
| 1-75 | 0 | Cl | O | CHMe | OEt |
| 1-76 | 0 | Cl | O | CHMe | OCH₂Ph |
| 1-77 | 0 | Cl | O | CHMe | NH₂ |
| 1-78 | 0 | Cl | O | CHMe | NHEt |
| 1-79 | 0 | Cl | O | CHMe | NHSO₂Me |
| 1-80 | 0 | Cl | O | CHMe | NHSO₂NMe₂ |
| 1-81 | 0 | Cl | O | CHMe | NHSO₂N(Me)(CHMe₂) |
| 1-82 | 0 | H | NH | CHMe | OH |
| 1-83 | 0 | H | NH | CHMe | OMe |
| 1-84 | 0 | H | NH | CHMe | OEt |
| 1-85 | 0 | H | NH | CHMe | OCH₂Ph |
| 1-86 | 0 | H | NH | CHMe | NH₂ |
| 1-87 | 0 | H | NH | CHMe | NHEt |
| 1-88 | 0 | H | NH | CHMe | NHSO₂Me |
| 1-89 | 0 | H | NH | CHMe | NHSO₂NMe₂ |
| 1-90 | 0 | H | NH | CHMe | NHSO₂N(Me)(CHMe₂) |
| 1-91 | 0 | F | NH | CHMe | OH |
| 1-92 | 0 | F | NH | CHMe | OMe |
| 1-93 | 0 | F | NH | CHMe | OEt |
| 1-94 | 0 | F | NH | CHMe | OCH₂Ph |
| 1-95 | 0 | F | NH | CHMe | NH₂ |
| 1-96 | 0 | F | NH | CHMe | NHEt |
| 1-97 | 0 | F | NH | CHMe | NHSO₂Me |
| 1-98 | 0 | F | NH | CHMe | NHSO₂NMe₂ |
| 1-99 | 0 | F | NH | CHMe | NHSO₂N(Me)(CHMe₂) |
| 1-100 | 0 | Cl | NH | CHMe | OH |
| 1-101 | 0 | Cl | NH | CHMe | OMe |
| 1-102 | 0 | Cl | NH | CHMe | OEt |
| 1-103 | 0 | Cl | NH | CHMe | OCH₂Ph |
| 1-104 | 0 | Cl | NH | CHMe | NH₂ |
| 1-105 | 0 | Cl | NH | CHMe | NHEt |
| 1-106 | 0 | Cl | NH | CHMe | NHSO₂Me |
| 1-107 | 0 | Cl | NH | CHMe | NHSO₂NMe₂ |
| 1-108 | 0 | Cl | NH | CHMe | NHSO₂N(Me)(CHMe₂) |
| 1-109 | 0 | H | O | CHEt | OH |
| 1-110 | 0 | H | O | CHEt | OMe |
| 1-111 | 0 | H | O | CHEt | OEt |
| 1-112 | 0 | H | O | CHEt | OCH₂Ph |
| 1-113 | 0 | H | O | CHEt | NHSO₂Me |
| 1-114 | 0 | H | O | CHEt | NHSO₂N(Me)(CHMe₂) |
| 1-115 | 0 | F | O | CHEt | OH |
| 1-116 | 0 | F | O | CHEt | OMe |
| 1-117 | 0 | F | O | CHEt | OEt |
| 1-118 | 0 | F | O | CHEt | OCH₂Ph |
| 1-119 | 0 | F | O | CHEt | NHSO₂Me |
| 1-120 | 0 | F | O | CHEt | NHSO₂N(Me)(CHMe₂) |
| 1-121 | 0 | H | NH | CHEt | OH |
| 1-122 | 0 | H | NH | CHEt | OMe |
| 1-123 | 0 | H | NH | CHEt | OEt |
| 1-124 | 0 | H | NH | CHEt | OCH₂Ph |
| 1-125 | 0 | H | NH | CHEt | NHSO₂Me |
| 1-126 | 0 | H | NH | CHEt | NHSO₂N(Me)(CHMe₂) |
| 1-127 | 0 | F | NH | CHEt | OH |
| 1-128 | 0 | F | NH | CHEt | OMe |
| 1-129 | 0 | F | NH | CHEt | OEt |
| 1-130 | 0 | F | NH | CHEt | OCH₂Ph |
| 1-131 | 0 | F | NH | CHEt | NHSO₂Me |
| 1-132 | 0 | F | NH | CHEt | NHSO₂N(Me)(CHMe₂) |
| 1-133 | 0 | H | O | C(Me)₂ | OH |
| 1-134 | 0 | H | O | C(Me)₂ | OMe |
| 1-135 | 0 | H | O | C(Me)₂ | OEt |
| 1-136 | 0 | H | O | C(Me)₂ | OCH₂Ph |
| 1-137 | 0 | H | O | C(Me)₂ | NH₂ |
| 1-138 | 0 | H | O | C(Me)₂ | NHEt |
| 1-139 | 0 | H | O | C(Me)₂ | NHSO₂Me |
| 1-140 | 0 | H | O | C(Me)₂ | NHSO₂NMe₂ |
| 1-141 | 0 | H | O | C(Me)₂ | NHSO₂N(Me)(CHMe₂) |
| 1-142 | 0 | F | O | C(Me)₂ | OH |
| 1-143 | 0 | F | O | C(Me)₂ | OMe |
| 1-144 | 0 | F | O | C(Me)₂ | OEt |
| 1-145 | 0 | F | O | C(Me)₂ | OCH₂Ph |
| 1-146 | 0 | F | O | C(Me)₂ | NH₂ |
| 1-147 | 0 | F | O | C(Me)₂ | NHEt |
| 1-148 | 0 | F | O | C(Me)₂ | NHSO₂Me |
| 1-149 | 0 | F | O | C(Me)₂ | NHSO₂NMe₂ |
| 1-150 | 0 | F | O | C(Me)₂ | NHSO₂N(Me)(CHMe₂) |
| 1-151 | 0 | Cl | O | C(Me)₂ | OH |
| 1-152 | 0 | Cl | O | C(Me)₂ | OMe |
| 1-153 | 0 | Cl | O | C(Me)₂ | OEt |
| 1-154 | 0 | Cl | O | C(Me)₂ | OCH₂Ph |
| 1-155 | 0 | Cl | O | C(Me)₂ | NH₂ |
| 1-156 | 0 | Cl | O | C(Me)₂ | NHEt |
| 1-157 | 0 | Cl | O | C(Me)₂ | NHSO₂Me |
| 1-158 | 0 | Cl | O | C(Me)₂ | NHSO₂NMe₂ |
| 1-159 | 0 | Cl | O | C(Me)₂ | NHSO₂N(Me)(CHMe₂) |
| 1-160 | 0 | H | NH | C(Me)₂ | OH |
| 1-161 | 0 | H | NH | C(Me)₂ | OMe |
| 1-162 | 0 | H | NH | C(Me)₂ | OEt |
| 1-163 | 0 | H | NH | C(Me)₂ | OCH₂Ph |
| 1-164 | 0 | H | NH | C(Me)₂ | NH₂ |
| 1-165 | 0 | H | NH | C(Me)₂ | NHEt |
| 1-166 | 0 | H | NH | C(Me)₂ | NHSO₂Me |
| 1-167 | 0 | H | NH | C(Me)₂ | NHSO₂NMe₂ |
| 1-168 | 0 | H | NH | C(Me)₂ | NHSO₂N(Me)(CHMe₂) |
| 1-169 | 0 | F | NH | C(Me)₂ | OH |
| 1-170 | 0 | F | NH | C(Me)₂ | OMe |
| 1-171 | 0 | F | NH | C(Me)₂ | OEt |
| 1-172 | 0 | F | NH | C(Me)₂ | OCH₂Ph |
| 1-173 | 0 | F | NH | C(Me)₂ | NH₂ |
| 1-174 | 0 | F | NH | C(Me)₂ | NHEt |
| 1-175 | 0 | F | NH | C(Me)₂ | NHSO₂Me |
| 1-176 | 0 | F | NH | C(Me)₂ | NHSO₂NMe₂ |
| 1-177 | 0 | F | NH | C(Me)₂ | NHSO₂N(Me)(CHMe₂) |
| 1-178 | 0 | Cl | NH | C(Me)₂ | OH |
| 1-179 | 0 | Cl | NH | C(Me)₂ | OMe |
| 1-180 | 0 | Cl | NH | C(Me)₂ | OEt |
| 1-181 | 0 | Cl | NH | C(Me)₂ | OCH₂Ph |
| 1-182 | 0 | Cl | NH | C(Me)₂ | NH₂ |
| 1-183 | 0 | Cl | NH | C(Me)₂ | NHEt |
| 1-184 | 0 | Cl | NH | C(Me)₂ | NHSO₂Me |
| 1-185 | 0 | Cl | NH | C(Me)₂ | NHSO₂NMe₂ |
| 1-186 | 0 | Cl | NH | C(Me)₂ | NHSO₂N(Me)(CHMe₂) |
| 1-187 | 0 | H | O | C(CH₂CH₂) | OH |
| 1-188 | 0 | H | O | C(CH₂CH₂) | OMe |
| 1-189 | 0 | H | O | C(CH₂CH₂) | OEt |
| 1-190 | 0 | H | O | C(CH₂CH₂) | OCH₂Ph |
| 1-191 | 0 | H | O | C(CH₂CH₂) | NHSO₂Me |
| 1-192 | 0 | H | O | C(CH₂CH₂) | NHSO₂N(Me)(CHMe₂) |
| 1-193 | 0 | F | O | C(CH₂CH₂) | OH |
| 1-194 | 0 | F | O | C(CH₂CH₂) | OMe |
| 1-195 | 0 | F | O | C(CH₂CH₂) | OEt |
| 1-196 | 0 | F | O | C(CH₂CH₂) | OCH₂Ph |
| 1-197 | 0 | F | O | C(CH₂CH₂) | NHSO₂Me |
| 1-198 | 0 | F | O | C(CH₂CH₂) | NHSO₂N(Me)(CHMe₂) |
| 1-199 | 0 | H | NH | C(CH₂CH₂) | OH |
| 1-200 | 0 | H | NH | C(CH₂CH₂) | OMe |
| 1-201 | 0 | H | NH | C(CH₂CH₂) | OEt |
| 1-202 | 0 | H | NH | C(CH₂CH₂) | OCH₂Ph |
| 1-203 | 0 | H | NH | C(CH₂CH₂) | NHSO₂Me |
| 1-204 | 0 | H | NH | C(CH₂CH₂) | NHSO₂N(Me)(CHMe₂) |
| 1-205 | 0 | F | NH | C(CH₂CH₂) | OH |
| 1-206 | 0 | F | NH | C(CH₂CH₂) | OMe |
| 1-207 | 0 | F | NH | C(CH₂CH₂) | OEt |
| 1-208 | 0 | F | NH | C(CH₂CH₂) | OCH₂Ph |
| 1-209 | 0 | F | NH | C(CH₂CH₂) | NHSO₂Me |
| 1-210 | 0 | F | NH | C(CH₂CH₂) | NHSO₂N(Me)(CHMe₂) |
| 1-211 | 0 | H | O | C(CH₂CH₂OCH₂CH₂) | OH |
| 1-212 | 0 | H | O | C(CH₂CH₂OCH₂CH₂) | OMe |
| 1-213 | 0 | H | O | C(CH₂CH₂OCH₂CH₂) | OEt |
| 1-214 | 0 | H | O | C(CH₂CH₂OCH₂CH₂) | OCH₂Ph |
| 1-215 | 0 | H | O | C(CH₂CH₂OCH₂CH₂) | NHSO₂Me |
| 1-216 | 0 | H | O | C(CH₂CH₂OCH₂CH₂) | NHSO₂N(Me)(CHMe₂) |
| 1-217 | 0 | F | O | C(CH₂CH₂OCH₂CH₂) | OH |
| 1-218 | 0 | F | O | C(CH₂CH₂OCH₂CH₂) | OMe |
| 1-219 | 0 | F | O | C(CH₂CH₂OCH₂CH₂) | OEt |
| 1-220 | 0 | F | O | C(CH₂CH₂OCH₂CH₂) | OCH₂Ph |
| 1-221 | 0 | F | O | C(CH₂CH₂OCH₂CH₂) | NHSO₂Me |
| 1-222 | 0 | F | O | C(CH₂CH₂OCH₂CH₂) | NHSO₂N(Me)(CHMe₂) |
| 1-223 | 0 | H | NH | C(CH₂CH₂OCH₂CH₂) | OH |
| 1-224 | 0 | H | NH | C(CH₂CH₂OCH₂CH₂) | OMe |
| 1-225 | 0 | H | NH | C(CH₂CH₂OCH₂CH₂) | OEt |
| 1-226 | 0 | H | NH | C(CH₂CH₂OCH₂CH₂) | OCH₂Ph |
| 1-227 | 0 | H | NH | C(CH₂CH₂OCH₂CH₂) | NHSO₂Me |
| 1-228 | 0 | H | NH | C(CH₂CH₂OCH₂CH₂) | NHSO₂N(Me)(CHMe₂) |
| 1-229 | 0 | F | NH | C(CH₂CH₂OCH₂CH₂) | OH |
| 1-230 | 0 | F | NH | C(CH₂CH₂OCH₂CH₂) | OMe |
| 1-231 | 0 | F | NH | C(CH₂CH₂OCH₂CH₂) | OEt |
| 1-232 | 0 | F | NH | C(CH₂CH₂OCH₂CH₂) | OCH₂Ph |
| 1-233 | 0 | F | NH | C(CH₂CH₂OCH₂CH₂) | NHSO₂Me |
| 1-234 | 0 | F | NH | C(CH₂CH₂OCH₂CH₂) | NHSO₂N(Me)(CHMe₂) |
| 1-235 | 0 | H | O | CH₂CH₂ | OH |
| 1-236 | 0 | H | O | CH₂CH₂ | OMe |
| 1-237 | 0 | H | O | CH₂CH₂ | OEt |
| 1-238 | 0 | H | O | CH₂CH₂ | OCH₂Ph |
| 1-239 | 0 | H | O | CH₂CH₂ | NH₂ |
| 1-240 | 0 | H | O | CH₂CH₂ | NHEt |
| 1-241 | 0 | H | O | CH₂CH₂ | NHSO₂Me |
| 1-242 | 0 | H | O | CH₂CH₂ | NHSO₂NMe₂ |
| 1-243 | 0 | H | O | CH₂CH₂ | NHSO₂N(Me)(CHMe₂) |
| 1-244 | 0 | F | O | CH₂CH₂ | OH |
| 1-245 | 0 | F | O | CH₂CH₂ | OMe |
| 1-246 | 0 | F | O | CH₂CH₂ | OEt |
| 1-247 | 0 | F | O | CH₂CH₂ | OCH₂Ph |
| 1-248 | 0 | F | O | CH₂CH₂ | NH₂ |
| 1-249 | 0 | F | O | CH₂CH₂ | NHEt |
| 1-250 | 0 | F | O | CH₂CH₂ | NHSO₂Me |
| 1-251 | 0 | F | O | CH₂CH₂ | NHSO₂NMe₂ |
| 1-252 | 0 | F | O | CH₂CH₂ | NHSO₂N(Me)(CHMe₂) |
| 1-253 | 0 | Cl | O | CH₂CH₂ | OH |
| 1-254 | 0 | Cl | O | CH₂CH₂ | OMe |
| 1-255 | 0 | Cl | O | CH₂CH₂ | OEt |
| 1-256 | 0 | Cl | O | CH₂CH₂ | OCH₂Ph |
| 1-257 | 0 | Cl | O | CH₂CH₂ | NH₂ |
| 1-258 | 0 | Cl | O | CH₂CH₂ | NHEt |
| 1-259 | 0 | Cl | O | CH₂CH₂ | NHSO₂Me |
| 1-260 | 0 | Cl | O | CH₂CH₂ | NHSO₂NMe₂ |
| 1-261 | 0 | Cl | O | CH₂CH₂ | NHSO₂N(Me)(CHMe₂) |
| 1-262 | 0 | H | NH | CH₂CH₂ | OH |
| 1-263 | 0 | H | NH | CH₂CH₂ | OMe |
| 1-264 | 0 | H | NH | CH₂CH₂ | OEt |
| 1-265 | 0 | H | NH | CH₂CH₂ | OCH₂Ph |
| 1-266 | 0 | H | NH | CH₂CH₂ | NH₂ |
| 1-267 | 0 | H | NH | CH₂CH₂ | NHEt |
| 1-268 | 0 | H | NH | CH₂CH₂ | NHSO₂Me |
| 1-269 | 0 | H | NH | CH₂CH₂ | NHSO₂NMe₂ |
| 1-270 | 0 | H | NH | CH₂CH₂ | NHSO₂N(Me)(CHMe₂) |
| 1-271 | 0 | F | NH | CH₂CH₂ | OH |
| 1-272 | 0 | F | NH | CH₂CH₂ | OMe |
| 1-273 | 0 | F | NH | CH₂CH₂ | OEt |
| 1-274 | 0 | F | NH | CH₂CH₂ | OCH₂Ph |
| 1-275 | 0 | F | NH | CH₂CH₂ | NH₂ |
| 1-276 | 0 | F | NH | CH₂CH₂ | NHEt |
| 1-277 | 0 | F | NH | CH₂CH₂ | NHSO₂Me |
| 1-278 | 0 | F | NH | CH₂CH₂ | NHSO₂NMe₂ |
| 1-279 | 0 | F | NH | CH₂CH₂ | NHSO₂N(Me)(CHMe₂) |
| 1-280 | 0 | Cl | NH | CH₂CH₂ | OH |
| 1-281 | 0 | Cl | NH | CH₂CH₂ | OMe |
| 1-282 | 0 | Cl | NH | CH₂CH₂ | OEt |
| 1-283 | 0 | Cl | NH | CH₂CH₂ | OCH₂Ph |
| 1-284 | 0 | Cl | NH | CH₂CH₂ | NH₂ |
| 1-285 | 0 | Cl | NH | CH₂CH₂ | NHEt |
| 1-286 | 0 | Cl | NH | CH₂CH₂ | NHSO₂Me |
| 1-287 | 0 | Cl | NH | CH₂CH₂ | NHSO₂NMe₂ |
| 1-288 | 0 | Cl | NH | CH₂CH₂ | NHSO₂N(Me)(CHMe₂) |
| 1-289 | 0 | H | O | CHMeCH₂ | OH |
| 1-290 | 0 | H | O | CHMeCH₂ | OMe |
| 1-291 | 0 | H | O | CHMeCH₂ | OEt |
| 1-292 | 0 | H | O | CHMeCH₂ | OCH₂Ph |
| 1-293 | 0 | H | O | CHMeCH₂ | NH₂ |
| 1-294 | 0 | H | O | CHMeCH₂ | NHEt |
| 1-295 | 0 | H | O | CHMeCH₂ | NHSO₂Me |
| 1-296 | 0 | H | O | CHMeCH₂ | NHSO₂NMe₂ |
| 1-297 | 0 | H | O | CHMeCH₂ | NHSO₂N(Me)(CHMe₂) |
| 1-298 | 0 | F | O | CHMeCH₂ | OH |
| 1-299 | 0 | F | O | CHMeCH₂ | OMe |
| 1-300 | 0 | F | O | CHMeCH₂ | OEt |
| 1-301 | 0 | F | O | CHMeCH₂ | OCH₂Ph |
| 1-302 | 0 | F | O | CHMeCH₂ | NH₂ |
| 1-303 | 0 | F | O | CHMeCH₂ | NHEt |
| 1-304 | 0 | F | O | CHMeCH₂ | NHSO₂Me |
| 1-305 | 0 | F | O | CHMeCH₂ | NHSO₂NMe₂ |
| 1-306 | 0 | F | O | CHMeCH₂ | NHSO₂N(Me)(CHMe₂) |
| 1-307 | 0 | Cl | O | CHMeCH₂ | OH |
| 1-308 | 0 | Cl | O | CHMeCH₂ | OMe |
| 1-309 | 0 | Cl | O | CHMeCH₂ | OEt |
| 1-310 | 0 | Cl | O | CHMeCH₂ | OCH₂Ph |
| 1-311 | 0 | Cl | O | CHMeCH₂ | NH₂ |
| 1-312 | 0 | Cl | O | CHMeCH₂ | NHEt |
| 1-313 | 0 | Cl | O | CHMeCH₂ | NHSO₂Me |
| 1-314 | 0 | Cl | O | CHMeCH₂ | NHSO₂NMe₂ |
| 1-315 | 0 | Cl | O | CHMeCH₂ | NHSO₂N(Me)(CHMe₂) |
| 1-316 | 0 | H | NH | CHMeCH₂ | OH |
| 1-317 | 0 | H | NH | CHMeCH₂ | OMe |
| 1-318 | 0 | H | NH | CHMeCH₂ | OEt |
| 1-319 | 0 | H | NH | CHMeCH₂ | OCH₂Ph |
| 1-320 | 0 | H | NH | CHMeCH₂ | NH₂ |
| 1-321 | 0 | H | NH | CHMeCH₂ | NHEt |
| 1-322 | 0 | H | NH | CHMeCH₂ | NHSO₂Me |
| 1-323 | 0 | H | NH | CHMeCH₂ | NHSO₂NMe₂ |
| 1-324 | 0 | H | NH | CHMeCH₂ | NHSO₂N(Me)(CHMe₂) |
| 1-325 | 0 | F | NH | CHMeCH₂ | OH |
| 1-326 | 0 | F | NH | CHMeCH₂ | OMe |
| 1-327 | 0 | F | NH | CHMeCH₂ | OEt |
| 1-328 | 0 | F | NH | CHMeCH₂ | OCH₂Ph |
| 1-329 | 0 | F | NH | CHMeCH₂ | NH₂ |
| 1-330 | 0 | F | NH | CHMeCH₂ | NHEt |
| 1-331 | 0 | F | NH | CHMeCH₂ | NHSO₂Me |
| 1-332 | 0 | F | NH | CHMeCH₂ | NHSO₂NMe₂ |
| 1-333 | 0 | F | NH | CHMeCH₂ | NHSO₂N(Me)(CHMe₂) |
| 1-334 | 0 | Cl | NH | CHMeCH₂ | OH |
| 1-335 | 0 | Cl | NH | CHMeCH₂ | OMe |
| 1-336 | 0 | Cl | NH | CHMeCH₂ | OEt |
| 1-337 | 0 | Cl | NH | CHMeCH₂ | OCH₂Ph |
| 1-338 | 0 | Cl | NH | CHMeCH₂ | NH₂ |
| 1-339 | 0 | Cl | NH | CHMeCH₂ | NHEt |
| 1-340 | 0 | Cl | NH | CHMeCH₂ | NHSO₂Me |
| 1-341 | 0 | Cl | NH | CHMeCH₂ | NHSO₂NMe₂ |
| 1-342 | 0 | Cl | NH | CHMeCH₂ | NHSO₂N(Me)(CHMe₂) |
| 1-343 | 0 | H | O | CHEtCH₂ | OH |
| 1-344 | 0 | H | O | CHEtCH₂ | OMe |
| 1-345 | 0 | H | O | CHEtCH₂ | OEt |
| 1-346 | 0 | H | O | CHEtCH₂ | OCH₂Ph |
| 1-347 | 0 | H | O | CHEtCH₂ | NHSO₂Me |
| 1-348 | 0 | H | O | CHEtCH₂ | NHSO₂N(Me)(CHMe₂) |
| 1-349 | 0 | F | O | CHEtCH₂ | OH |
| 1-350 | 0 | F | O | CHEtCH₂ | OMe |
| 1-351 | 0 | F | O | CHEtCH₂ | OEt |
| 1-352 | 0 | F | O | CHEtCH₂ | OCH₂Ph |
| 1-353 | 0 | F | O | CHEtCH₂ | NHSO₂Me |
| 1-354 | 0 | F | O | CHEtCH₂ | NHSO₂N(Me)(CHMe₂) |
| 1-355 | 0 | H | NH | CHEtCH₂ | OH |
| 1-356 | 0 | H | NH | CHEtCH₂ | OMe |
| 1-357 | 0 | H | NH | CHEtCH₂ | OEt |
| 1-358 | 0 | H | NH | CHEtCH₂ | OCH₂Ph |
| 1-359 | 0 | H | NH | CHEtCH₂ | NHSO₂Me |
| 1-360 | 0 | H | NH | CHEtCH₂ | NHSO₂N(Me)(CHMe₂) |
| 1-361 | 0 | F | NH | CHEtCH₂ | OH |
| 1-362 | 0 | F | NH | CHEtCH₂ | OMe |
| 1-363 | 0 | F | NH | CHEtCH₂ | OEt |
| 1-364 | 0 | F | NH | CHEtCH₂ | OCH₂Ph |
| 1-365 | 0 | F | NH | CHEtCH₂ | NHSO₂Me |
| 1-366 | 0 | F | NH | CHEtCH₂ | NHSO₂N(Me)(CHMe₂) |
| 1-367 | 0 | H | O | CH₂CHMe | OH |
| 1-368 | 0 | H | O | CH₂CHMe | OMe |
| 1-369 | 0 | H | O | CH₂CHMe | OEt |
| 1-370 | 0 | H | O | CH₂CHMe | OCH₂Ph |
| 1-371 | 0 | H | O | CH₂CHMe | NH₂ |
| 1-372 | 0 | H | O | CH₂CHMe | NHEt |
| 1-373 | 0 | H | O | CH₂CHMe | NHSO₂Me |
| 1-374 | 0 | H | O | CH₂CHMe | NHSO₂NMe₂ |
| 1-375 | 0 | H | O | CH₂CHMe | NHSO₂N(Me)(CHMe₂) |
| 1-376 | 0 | F | O | CH₂CHMe | OH |
| 1-377 | 0 | F | O | CH₂CHMe | OMe |
| 1-378 | 0 | F | O | CH₂CHMe | OEt |
| 1-379 | 0 | F | O | CH₂CHMe | OCH₂Ph |
| 1-380 | 0 | F | O | CH₂CHMe | NH₂ |
| 1-381 | 0 | F | O | CH₂CHMe | NHEt |
| 1-382 | 0 | F | O | CH₂CHMe | NHSO₂Me |
| 1-383 | 0 | F | O | CH₂CHMe | NHSO₂NMe₂ |
| 1-384 | 0 | F | O | CH₂CHMe | NHSO₂N(Me)(CHMe₂) |
| 1-385 | 0 | Cl | O | CH₂CHMe | OH |
| 1-386 | 0 | Cl | O | CH₂CHMe | OMe |
| 1-387 | 0 | Cl | O | CH₂CHMe | OEt |
| 1-388 | 0 | Cl | O | CH₂CHMe | OCH₂Ph |
| 1-389 | 0 | Cl | O | CH₂CHMe | NH₂ |
| 1-390 | 0 | Cl | O | CH₂CHMe | NHEt |
| 1-391 | 0 | Cl | O | CH₂CHMe | NHSO₂Me |
| 1-392 | 0 | Cl | O | CH₂CHMe | NHSO₂NMe₂ |
| 1-393 | 0 | Cl | O | CH₂CHMe | NHSO₂N(Me)(CHMe₂) |
| 1-394 | 0 | H | NH | CH₂CHMe | OH |
| 1-395 | 0 | H | NH | CH₂CHMe | OMe |
| 1-396 | 0 | H | NH | CH₂CHMe | OEt |
| 1-397 | 0 | H | NH | CH₂CHMe | OCH₂Ph |
| 1-398 | 0 | H | NH | CH₂CHMe | NH₂ |
| 1-399 | 0 | H | NH | CH₂CHMe | NHEt |
| 1-400 | 0 | H | NH | CH₂CHMe | NHSO₂Me |
| 1-401 | 0 | H | NH | CH₂CHMe | NHSO₂NMe₂ |
| 1-402 | 0 | H | NH | CH₂CHMe | NHSO₂N(Me)(CHMe₂) |
| 1-403 | 0 | F | NH | CH₂CHMe | OH |
| 1-404 | 0 | F | NH | CH₂CHMe | OMe |
| 1-405 | 0 | F | NH | CH₂CHMe | OEt |
| 1-406 | 0 | F | NH | CH₂CHMe | OCH₂Ph |
| 1-407 | 0 | F | NH | CH₂CHMe | NH₂ |
| 1-408 | 0 | F | NH | CH₂CHMe | NHEt |
| 1-409 | 0 | F | NH | CH₂CHMe | NHSO₂Me |
| 1-410 | 0 | F | NH | CH₂CHMe | NHSO₂NMe₂ |
| 1-411 | 0 | F | NH | CH₂CHMe | NHSO₂N(Me)(CHMe₂) |
| 1-412 | 0 | Cl | NH | CH₂CHMe | OH |
| 1-413 | 0 | Cl | NH | CH₂CHMe | OMe |
| 1-414 | 0 | Cl | NH | CH₂CHMe | OEt |
| 1-415 | 0 | Cl | NH | CH₂CHMe | OCH₂Ph |
| 1-416 | 0 | Cl | NH | CH₂CHMe | NH₂ |
| 1-417 | 0 | Cl | NH | CH₂CHMe | NHEt |
| 1-418 | 0 | Cl | NH | CH₂CHMe | NHSO₂Me |
| 1-419 | 0 | Cl | NH | CH₂CHMe | NHSO₂NMe₂ |
| 1-420 | 0 | Cl | NH | CH₂CHMe | NHSO₂N(Me)(CHMe₂) |
| 1-421 | 0 | H | O | CHMeCHMe | OH |
| 1-422 | 0 | H | O | CHMeCHMe | OMe |
| 1-423 | 0 | H | O | CHMeCHMe | OEt |
| 1-424 | 0 | H | O | CHMeCHMe | OCH₂Ph |
| 1-425 | 0 | H | O | CHMeCHMe | NHSO₂Me |
| 1-426 | 0 | H | O | CHMeCHMe | NHSO₂N(Me)(CHMe₂) |
| 1-427 | 0 | F | O | CHMeCHMe | OH |
| 1-428 | 0 | F | O | CHMeCHMe | OMe |
| 1-429 | 0 | F | O | CHMeCHMe | OEt |
| 1-430 | 0 | F | O | CHMeCHMe | OCH₂Ph |
| 1-431 | 0 | F | O | CHMeCHMe | NHSO₂Me |
| 1-432 | 0 | F | O | CHMeCHMe | NHSO₂N(Me)(CHMe₂) |
| 1-433 | 0 | H | NH | CHMeCHMe | OH |
| 1-434 | 0 | H | NH | CHMeCHMe | OMe |
| 1-435 | 0 | H | NH | CHMeCHMe | OEt |
| 1-436 | 0 | H | NH | CHMeCHMe | OCH₂Ph |
| 1-437 | 0 | H | NH | CHMeCHMe | NHSO₂Me |
| 1-438 | 0 | H | NH | CHMeCHMe | NHSO₂N(Me)(CHMe₂) |
| 1-439 | 0 | F | NH | CHMeCHMe | OH |
| 1-440 | 0 | F | NH | CHMeCHMe | OMe |
| 1-441 | 0 | F | NH | CHMeCHMe | OEt |
| 1-442 | 0 | F | NH | CHMeCHMe | OCH₂Ph |
| 1-443 | 0 | F | NH | CHMeCHMe | NHSO₂Me |
| 1-444 | 0 | F | NH | CHMeCHMe | NHSO₂N(Me)(CHMe₂) |
| 1-445 | 0 | H | O | CMe₂CH₂ | OH |
| 1-446 | 0 | H | O | CMe₂CH₂ | OMe |
| 1-447 | 0 | H | O | CMe₂CH₂ | OEt |
| 1-448 | 0 | H | O | CMe₂CH₂ | OCH₂Ph |
| 1-449 | 0 | H | O | CMe₂CH₂ | NH₂ |
| 1-450 | 0 | H | O | CMe₂CH₂ | NHEt |
| 1-451 | 0 | H | O | CMe₂CH₂ | NHSO₂Me |
| 1-452 | 0 | H | O | CMe₂CH₂ | NHSO₂NMe₂ |
| 1-453 | 0 | H | O | CMe₂CH₂ | NHSO₂N(Me)(CHMe₂) |
| 1-454 | 0 | F | O | CMe₂CH₂ | OH |
| 1-455 | 0 | F | O | CMe₂CH₂ | OMe |
| 1-456 | 0 | F | O | CMe₂CH₂ | OEt |
| 1-457 | 0 | F | O | CMe₂CH₂ | OCH₂Ph |
| 1-458 | 0 | F | O | CMe₂CH₂ | NH₂ |
| 1-459 | 0 | F | O | CMe₂CH₂ | NHEt |
| 1-460 | 0 | F | O | CMe₂CH₂ | NHSO₂Me |
| 1-461 | 0 | F | O | CMe₂CH₂ | NHSO₂NMe₂ |
| 1-462 | 0 | F | O | CMe₂CH₂ | NHSO₂N(Me)(CHMe₂) |
| 1-463 | 0 | Cl | O | CMe₂CH₂ | OH |
| 1-464 | 0 | Cl | O | CMe₂CH₂ | OMe |
| 1-465 | 0 | Cl | O | CMe₂CH₂ | OEt |
| 1-466 | 0 | Cl | O | CMe₂CH₂ | OCH₂Ph |
| 1-467 | 0 | Cl | O | CMe₂CH₂ | NH₂ |
| 1-468 | 0 | Cl | O | CMe₂CH₂ | NHEt |
| 1-469 | 0 | Cl | O | CMe₂CH₂ | NHSO₂Me |
| 1-470 | 0 | Cl | O | CMe₂CH₂ | NHSO₂NMe₂ |
| 1-471 | 0 | Cl | O | CMe₂CH₂ | NHSO₂N(Me)(CHMe₂) |
| 1-472 | 0 | H | NH | CMe₂CH₂ | OH |
| 1-473 | 0 | H | NH | CMe₂CH₂ | OMe |
| 1-474 | 0 | H | NH | CMe₂CH₂ | OEt |
| 1-475 | 0 | H | NH | CMe₂CH₂ | OCH₂Ph |
| 1-476 | 0 | H | NH | CMe₂CH₂ | NH₂ |
| 1-477 | 0 | H | NH | CMe₂CH₂ | NHEt |
| 1-478 | 0 | H | NH | CMe₂CH₂ | NHSO₂Me |
| 1-479 | 0 | H | NH | CMe₂CH₂ | NHSO₂NMe₂ |
| 1-480 | 0 | H | NH | CMe₂CH₂ | NHSO₂N(Me)(CHMe₂) |
| 1-481 | 0 | F | NH | CMe₂CH₂ | OH |
| 1-482 | 0 | F | NH | CMe₂CH₂ | OMe |
| 1-483 | 0 | F | NH | CMe₂CH₂ | OEt |
| 1-484 | 0 | F | NH | CMe₂CH₂ | OCH₂Ph |
| 1-485 | 0 | F | NH | CMe₂CH₂ | NH₂ |
| 1-486 | 0 | F | NH | CMe₂CH₂ | NHEt |
| 1-487 | 0 | F | NH | CMe₂CH₂ | NHSO₂Me |
| 1-488 | 0 | F | NH | CMe₂CH₂ | NHSO₂NMe₂ |
| 1-489 | 0 | F | NH | CMe₂CH₂ | NHSO₂N(Me)(CHMe₂) |
| 1-490 | 0 | Cl | NH | CMe₂CH₂ | OH |
| 1-491 | 0 | Cl | NH | CMe₂CH₂ | OMe |
| 1-492 | 0 | Cl | NH | CMe₂CH₂ | OEt |
| 1-493 | 0 | Cl | NH | CMe₂CH₂ | OCH₂Ph |
| 1-494 | 0 | Cl | NH | CMe₂CH₂ | NH₂ |
| 1-495 | 0 | Cl | NH | CMe₂CH₂ | NHEt |
| 1-496 | 0 | Cl | NH | CMe₂CH₂ | NHSO₂Me |
| 1-497 | 0 | Cl | NH | CMe₂CH₂ | NHSO₂NMe₂ |
| 1-498 | 0 | Cl | NH | CMe₂CH₂ | NHSO₂N(Me)(CHMe₂) |
| 1-499 | 0 | H | O | CH₂CMe₂ | OH |
| 1-500 | 0 | H | O | CH₂CMe₂ | OMe |
| 1-501 | 0 | H | O | CH₂CMe₂ | OEt |
| 1-502 | 0 | H | O | CH₂CMe₂ | OCH₂Ph |
| 1-503 | 0 | H | O | CH₂CMe₂ | NH₂ |
| 1-504 | 0 | H | O | CH₂CMe₂ | NHEt |
| 1-505 | 0 | H | O | CH₂CMe₂ | NHSO₂Me |
| 1-506 | 0 | H | O | CH₂CMe₂ | NHSO₂NMe₂ |
| 1-507 | 0 | H | O | CH₂CMe₂ | NHSO₂N(Me)(CHMe₂) |
| 1-508 | 0 | F | O | CH₂CMe₂ | OH |
| 1-509 | 0 | F | O | CH₂CMe₂ | OMe |
| 1-510 | 0 | F | O | CH₂CMe₂ | OEt |
| 1-511 | 0 | F | O | CH₂CMe₂ | OCH₂Ph |
| 1-512 | 0 | F | O | CH₂CMe₂ | NH₂ |
| 1-513 | 0 | F | O | CH₂CMe₂ | NHEt |
| 1-514 | 0 | F | O | CH₂CMe₂ | NHSO₂Me |
| 1-515 | 0 | F | O | CH₂CMe₂ | NHSO₂NMe₂ |
| 1-516 | 0 | F | O | CH₂CMe₂ | NHSO₂N(Me)(CHMe₂) |
| 1-517 | 0 | Cl | O | CH₂CMe₂ | OH |
| 1-518 | 0 | Cl | O | CH₂CMe₂ | OMe |
| 1-519 | 0 | Cl | O | CH₂CMe₂ | OEt |
| 1-520 | 0 | Cl | O | CH₂CMe₂ | OCH₂Ph |
| 1-521 | 0 | Cl | O | CH₂CMe₂ | NH₂ |
| 1-522 | 0 | Cl | O | CH₂CMe₂ | NHEt |
| 1-523 | 0 | Cl | O | CH₂CMe₂ | NHSO₂Me |
| 1-524 | 0 | Cl | O | CH₂CMe₂ | NHSO₂NMe₂ |
| 1-525 | 0 | Cl | O | CH₂CMe₂ | NHSO₂N(Me)(CHMe₂) |
| 1-526 | 0 | H | NH | CH₂CMe₂ | OH |
| 1-527 | 0 | H | NH | CH₂CMe₂ | OMe |
| 1-528 | 0 | H | NH | CH₂CMe₂ | OEt |
| 1-529 | 0 | H | NH | CH₂CMe₂ | OCH₂Ph |
| 1-530 | 0 | H | NH | CH₂CMe₂ | NH₂ |
| 1-531 | 0 | H | NH | CH₂CMe₂ | NHEt |
| 1-532 | 0 | H | NH | CH₂CMe₂ | NHSO₂Me |
| 1-533 | 0 | H | NH | CH₂CMe₂ | NHSO₂NMe₂ |
| 1-534 | 0 | H | NH | CH₂CMe₂ | NHSO₂N(Me)(CHMe₂) |
| 1-535 | 0 | F | NH | CH₂CMe₂ | OH |
| 1-536 | 0 | F | NH | CH₂CMe₂ | OMe |
| 1-537 | 0 | F | NH | CH₂CMe₂ | OEt |
| 1-538 | 0 | F | NH | CH₂CMe₂ | OCH₂Ph |
| 1-539 | 0 | F | NH | CH₂CMe₂ | NH₂ |
| 1-540 | 0 | F | NH | CH₂CMe₂ | NHEt |
| 1-541 | 0 | F | NH | CH₂CMe₂ | NHSO₂Me |
| 1-542 | 0 | F | NH | CH₂CMe₂ | NHSO₂NMe₂ |
| 1-543 | 0 | F | NH | CH₂CMe₂ | NHSO₂N(Me)(CHMe₂) |
| 1-544 | 0 | Cl | NH | CH₂CMe₂ | OH |
| 1-545 | 0 | Cl | NH | CH₂CMe₂ | OMe |
| 1-546 | 0 | Cl | NH | CH₂CMe₂ | OEt |
| 1-547 | 0 | Cl | NH | CH₂CMe₂ | OCH₂Ph |
| 1-548 | 0 | Cl | NH | CH₂CMe₂ | NH₂ |
| 1-549 | 0 | Cl | NH | CH₂CMe₂ | NHEt |
| 1-550 | 0 | Cl | NH | CH₂CMe₂ | NHSO₂Me |
| 1-551 | 0 | Cl | NH | CH₂CMe₂ | NHSO₂NMe₂ |
| 1-552 | 0 | Cl | NH | CH₂CMe₂ | NHSO₂N(Me)(CHMe₂) |
| 1-553 | 0 | H | O | CH₂CHCl | OH |
| 1-554 | 0 | H | O | CH₂CHCl | OMe |
| 1-555 | 0 | H | O | CH₂CHCl | OEt |
| 1-556 | 0 | H | O | CH₂CHCl | OCH₂Ph |
| 1-557 | 0 | H | O | CH₂CHCl | NHSO₂Me |
| 1-558 | 0 | H | O | CH₂CHCl | NHSO₂N(Me)(CHMe₂) |
| 1-559 | 0 | F | O | CH₂CHCl | OH |
| 1-560 | 0 | F | O | CH₂CHCl | OMe |
| 1-561 | 0 | F | O | CH₂CHCl | OEt |
| 1-562 | 0 | F | O | CH₂CHCl | OCH₂Ph |
| 1-563 | 0 | F | O | CH₂CHCl | NHSO₂Me |
| 1-564 | 0 | F | O | CH₂CHCl | NHSO₂N(Me)(CHMe₂) |
| 1-565 | 0 | H | NH | CH₂CHCl | OH |
| 1-566 | 0 | H | NH | CH₂CHCl | OMe |
| 1-567 | 0 | H | NH | CH₂CHCl | OEt |
| 1-568 | 0 | H | NH | CH₂CHCl | OCH₂Ph |
| 1-569 | 0 | H | NH | CH₂CHCl | NHSO₂Me |
| 1-570 | 0 | H | NH | CH₂CHCl | NHSO₂N(Me)(CHMe₂) |
| 1-571 | 0 | F | NH | CH₂CHCl | OH |
| 1-572 | 0 | F | NH | CH₂CHCl | OMe |
| 1-573 | 0 | F | NH | CH₂CHCl | OEt |
| 1-574 | 0 | F | NH | CH₂CHCl | OCH₂Ph |
| 1-575 | 0 | F | NH | CH₂CHCl | NHSO₂Me |
| 1-576 | 0 | F | NH | CH₂CHCl | NHSO₂N(Me)(CHMe₂) |
| 1-577 | 0 | H | O | C(CH₂CH₂)CH₂ | OH |
| 1-578 | 0 | H | O | C(CH₂CH₂)CH₂ | OMe |
| 1-579 | 0 | H | O | C(CH₂CH₂)CH₂ | OEt |
| 1-580 | 0 | H | O | C(CH₂CH₂)CH₂ | OCH₂Ph |
| 1-581 | 0 | H | O | C(CH₂CH₂)CH₂ | NHSO₂Me |
| 1-582 | 0 | H | O | C(CH₂CH₂)CH₂ | NHSO₂N(Me)(CHMe₂) |
| 1-583 | 0 | F | O | C(CH₂CH₂)CH₂ | OH |
| 1-584 | 0 | F | O | C(CH₂CH₂)CH₂ | OMe |
| 1-585 | 0 | F | O | C(CH₂CH₂)CH₂ | OEt |
| 1-586 | 0 | F | O | C(CH₂CH₂)CH₂ | OCH₂Ph |
| 1-587 | 0 | F | O | C(CH₂CH₂)CH₂ | NHSO₂Me |
| 1-588 | 0 | F | O | C(CH₂CH₂)CH₂ | NHSO₂N(Me)(CHMe₂) |
| 1-589 | 0 | H | NH | C(CH₂CH₂)CH₂ | OH |
| 1-590 | 0 | H | NH | C(CH₂CH₂)CH₂ | OMe |
| 1-591 | 0 | H | NH | C(CH₂CH₂)CH₂ | OEt |
| 1-592 | 0 | H | NH | C(CH₂CH₂)CH₂ | OCH₂Ph |
| 1-593 | 0 | H | NH | C(CH₂CH₂)CH₂ | NHSO₂Me |
| 1-594 | 0 | H | NH | C(CH₂CH₂)CH₂ | NHSO₂N(Me)(CHMe₂) |
| 1-595 | 0 | F | NH | C(CH₂CH₂)CH₂ | OH |
| 1-596 | 0 | F | NH | C(CH₂CH₂)CH₂ | OMe |
| 1-597 | 0 | F | NH | C(CH₂CH₂)CH₂ | OEt |
| 1-598 | 0 | F | NH | C(CH₂CH₂)CH₂ | OCH₂Ph |
| 1-599 | 0 | F | NH | C(CH₂CH₂)CH₂ | NHSO₂Me |
| 1-600 | 0 | F | NH | C(CH₂CH₂)CH₂ | NHSO₂N(Me)(CHMe₂) |
| 1-601 | 0 | H | O | CH₂C(CH₂CH₂) | OH |
| 1-602 | 0 | H | O | CH₂C(CH₂CH₂) | OMe |
| 1-603 | 0 | H | O | CH₂C(CH₂CH₂) | OEt |
| 1-604 | 0 | H | O | CH₂C(CH₂CH₂) | OCH₂Ph |
| 1-605 | 0 | H | O | CH₂C(CH₂CH₂) | NHSO₂Me |
| 1-606 | 0 | H | O | CH₂C(CH₂CH₂) | NHSO₂N(Me)(CHMe₂) |
| 1-607 | 0 | F | O | CH₂C(CH₂CH₂) | OH |
| 1-608 | 0 | F | O | CH₂C(CH₂CH₂) | OMe |
| 1-609 | 0 | F | O | CH₂C(CH₂CH₂) | OEt |
| 1-610 | 0 | F | O | CH₂C(CH₂CH₂) | OCH₂Ph |
| 1-611 | 0 | F | O | CH₂C(CH₂CH₂) | NHSO₂Me |
| 1-612 | 0 | F | O | CH₂C(CH₂CH₂) | NHSO₂N(Me)(CHMe₂) |
| 1-613 | 0 | H | NH | CH₂C(CH₂CH₂) | OH |
| 1-614 | 0 | H | NH | CH₂C(CH₂CH₂) | OMe |
| 1-615 | 0 | H | NH | CH₂C(CH₂CH₂) | OEt |
| 1-616 | 0 | H | NH | CH₂C(CH₂CH₂) | OCH₂Ph |
| 1-617 | 0 | H | NH | CH₂C(CH₂CH₂) | NHSO₂Me |
| 1-618 | 0 | H | NH | CH₂C(CH₂CH₂) | NHSO₂N(Me)(CHMe₂) |
| 1-619 | 0 | F | NH | CH₂C(CH₂CH₂) | OH |
| 1-620 | 0 | F | NH | CH₂C(CH₂CH₂) | OMe |
| 1-621 | 0 | F | NH | CH₂C(CH₂CH₂) | OEt |
| 1-622 | 0 | F | NH | CH₂C(CH₂CH₂) | OCH₂Ph |
| 1-623 | 0 | F | NH | CH₂C(CH₂CH₂) | NHSO₂Me |
| 1-624 | 0 | F | NH | CH₂C(CH₂CH₂) | NHSO₂N(Me)(CHMe₂) |
| 1-625 | 0 | H | O | CH(CH₂)CH | OH |
| 1-626 | 0 | H | O | CH(CH₂)CH | OMe |
| 1-627 | 0 | H | O | CH(CH₂)CH | OEt |
| 1-628 | 0 | H | O | CH(CH₂)CH | OCH₂Ph |
| 1-629 | 0 | H | O | CH(CH₂)CH | NHSO₂Me |
| 1-630 | 0 | H | O | CH(CH₂)CH | NHSO₂N(Me)(CHMe₂) |
| 1-631 | 0 | F | O | CH(CH₂)CH | OH |
| 1-632 | 0 | F | O | CH(CH₂)CH | OMe |
| 1-633 | 0 | F | O | CH(CH₂)CH | OEt |
| 1-634 | 0 | F | O | CH(CH₂)CH | OCH₂Ph |
| 1-635 | 0 | F | O | CH(CH₂)CH | NHSO₂Me |
| 1-636 | 0 | F | O | CH(CH₂)CH | NHSO₂N(Me)(CHMe₂) |
| 1-637 | 0 | H | NH | CH(CH₂)CH | OH |
| 1-638 | 0 | H | NH | CH(CH₂)CH | OMe |
| 1-639 | 0 | H | NH | CH(CH₂)CH | OEt |
| 1-640 | 0 | H | NH | CH(CH₂)CH | OCH₂Ph |
| 1-641 | 0 | H | NH | CH(CH₂)CH | NHSO₂Me |
| 1-642 | 0 | H | NH | CH(CH₂)CH | NHSO₂N(Me)(CHMe₂) |
| 1-643 | 0 | F | NH | CH(CH₂)CH | OH |
| 1-644 | 0 | F | NH | CH(CH₂)CH | OMe |
| 1-645 | 0 | F | NH | CH(CH₂)CH | OEt |
| 1-646 | 0 | F | NH | CH(CH₂)CH | OCH₂Ph |
| 1-647 | 0 | F | NH | CH(CH₂)CH | NHSO₂Me |
| 1-648 | 0 | F | NH | CH(CH₂)CH | NHSO₂N(Me)(CHMe₂) |
| 1-649 | 0 | H | O | CH₂CH₂CH₂ | OH |
| 1-650 | 0 | H | O | CH₂CH₂CH₂ | OMe |
| 1-651 | 0 | H | O | CH₂CH₂CH₂ | OEt |
| 1-652 | 0 | H | O | CH₂CH₂CH₂ | OCH₂Ph |
| 1-653 | 0 | H | O | CH₂CH₂CH₂ | NH₂ |
| 1-654 | 0 | H | O | CH₂CH₂CH₂ | NHEt |
| 1-655 | 0 | H | O | CH₂CH₂CH₂ | NHSO₂Me |
| 1-656 | 0 | H | O | CH₂CH₂CH₂ | NHSO₂NMe₂ |
| 1-657 | 0 | H | O | CH₂CH₂CH₂ | NHSO₂N(Me)(CHMe₂) |
| 1-658 | 0 | F | O | CH₂CH₂CH₂ | OH |
| 1-659 | 0 | F | O | CH₂CH₂CH₂ | OMe |
| 1-660 | 0 | F | O | CH₂CH₂CH₂ | OEt |
| 1-661 | 0 | F | O | CH₂CH₂CH₂ | OCH₂Ph |
| 1-662 | 0 | F | O | CH₂CH₂CH₂ | NH₂ |
| 1-663 | 0 | F | O | CH₂CH₂CH₂ | NHEt |
| 1-664 | 0 | F | O | CH₂CH₂CH₂ | NHSO₂Me |
| 1-665 | 0 | F | O | CH₂CH₂CH₂ | NHSO₂NMe₂ |
| 1-666 | 0 | F | O | CH₂CH₂CH₂ | NHSO₂N(Me)(CHMe₂) |
| 1-667 | 0 | Cl | O | CH₂CH₂CH₂ | OH |
| 1-668 | 0 | Cl | O | CH₂CH₂CH₂ | OMe |
| 1-669 | 0 | Cl | O | CH₂CH₂CH₂ | OEt |
| 1-670 | 0 | Cl | O | CH₂CH₂CH₂ | OCH₂Ph |
| 1-671 | 0 | Cl | O | CH₂CH₂CH₂ | NH₂ |
| 1-672 | 0 | Cl | O | CH₂CH₂CH₂ | NHEt |
| 1-673 | 0 | Cl | O | CH₂CH₂CH₂ | NHSO₂Me |
| 1-674 | 0 | Cl | O | CH₂CH₂CH₂ | NHSO₂NMe₂ |
| 1-675 | 0 | Cl | O | CH₂CH₂CH₂ | NHSO₂N(Me)(CHMe₂) |
| 1-676 | 0 | H | NH | CH₂CH₂CH₂ | OH |
| 1-677 | 0 | H | NH | CH₂CH₂CH₂ | OMe |
| 1-678 | 0 | H | NH | CH₂CH₂CH₂ | OEt |
| 1-679 | 0 | H | NH | CH₂CH₂CH₂ | OCH₂Ph |
| 1-680 | 0 | H | NH | CH₂CH₂CH₂ | NH₂ |
| 1-681 | 0 | H | NH | CH₂CH₂CH₂ | NHEt |
| 1-682 | 0 | H | NH | CH₂CH₂CH₂ | NHSO₂Me |
| 1-683 | 0 | H | NH | CH₂CH₂CH₂ | NHSO₂NMe₂ |
| 1-684 | 0 | H | NH | CH₂CH₂CH₂ | NHSO₂N(Me)(CHMe₂) |
| 1-685 | 0 | F | NH | CH₂CH₂CH₂ | OH |
| 1-686 | 0 | F | NH | CH₂CH₂CH₂ | OMe |
| 1-687 | 0 | F | NH | CH₂CH₂CH₂ | OEt |
| 1-688 | 0 | F | NH | CH₂CH₂CH₂ | OCH₂Ph |
| 1-689 | 0 | F | NH | CH₂CH₂CH₂ | NH₂ |
| 1-690 | 0 | F | NH | CH₂CH₂CH₂ | NHEt |
| 1-691 | 0 | F | NH | CH₂CH₂CH₂ | NHSO₂Me |
| 1-692 | 0 | F | NH | CH₂CH₂CH₂ | NHSO₂NMe₂ |
| 1-693 | 0 | F | NH | CH₂CH₂CH₂ | NHSO₂N(Me)(CHMe₂) |
| 1-694 | 0 | Cl | NH | CH₂CH₂CH₂ | OH |
| 1-695 | 0 | Cl | NH | CH₂CH₂CH₂ | OMe |
| 1-696 | 0 | Cl | NH | CH₂CH₂CH₂ | OEt |
| 1-697 | 0 | Cl | NH | CH₂CH₂CH₂ | OCH₂Ph |
| 1-698 | 0 | Cl | NH | CH₂CH₂CH₂ | NH₂ |
| 1-699 | 0 | Cl | NH | CH₂CH₂CH₂ | NHEt |
| 1-700 | 0 | Cl | NH | CH₂CH₂CH₂ | NHSO₂Me |
| 1-701 | 0 | Cl | NH | CH₂CH₂CH₂ | NHSO₂NMe₂ |
| 1-702 | 0 | Cl | NH | CH₂CH₂CH₂ | NHSO₂N(Me)(CHMe₂) |
| 1-703 | 0 | H | O | CHMeCH₂CH₂ | OH |
| 1-704 | 0 | H | O | CHMeCH₂CH₂ | OMe |
| 1-705 | 0 | H | O | CHMeCH₂CH₂ | OEt |
| 1-706 | 0 | H | O | CHMeCH₂CH₂ | OCH₂Ph |
| 1-707 | 0 | H | O | CHMeCH₂CH₂ | NHSO₂Me |
| 1-708 | 0 | H | O | CHMeCH₂CH₂ | NHSO₂N(Me)(CHMe₂) |
| 1-709 | 0 | F | O | CHMeCH₂CH₂ | OH |
| 1-710 | 0 | F | O | CHMeCH₂CH₂ | OMe |
| 1-711 | 0 | F | O | CHMeCH₂CH₂ | OEt |
| 1-712 | 0 | F | O | CHMeCH₂CH₂ | OCH₂Ph |
| 1-713 | 0 | F | O | CHMeCH₂CH₂ | NHSO₂Me |
| 1-714 | 0 | F | O | CHMeCH₂CH₂ | NHSO₂N(Me)(CHMe₂) |
| 1-715 | 0 | H | NH | CHMeCH₂CH₂ | OH |
| 1-716 | 0 | H | NH | CHMeCH₂CH₂ | OMe |
| 1-717 | 0 | H | NH | CHMeCH₂CH₂ | OEt |
| 1-718 | 0 | H | NH | CHMeCH₂CH₂ | OCH₂Ph |
| 1-719 | 0 | H | NH | CHMeCH₂CH₂ | NHSO₂Me |
| 1-720 | 0 | H | NH | CHMeCH₂CH₂ | NHSO₂N(Me)(CHMe₂) |
| 1-721 | 0 | F | NH | CHMeCH₂CH₂ | OH |
| 1-722 | 0 | F | NH | CHMeCH₂CH₂ | OMe |
| 1-723 | 0 | F | NH | CHMeCH₂CH₂ | OEt |
| 1-724 | 0 | F | NH | CHMeCH₂CH₂ | OCH₂Ph |
| 1-725 | 0 | F | NH | CHMeCH₂CH₂ | NHSO₂Me |
| 1-726 | 0 | F | NH | CHMeCH₂CH₂ | NHSO₂N(Me)(CHMe₂) |
| 1-727 | 0 | H | O | 1,3-cyclobutyl | OH |
| 1-728 | 0 | H | O | 1,3-cyclobutyl | OMe |
| 1-729 | 0 | H | O | 1,3-cyclobutyl | OEt |
| 1-730 | 0 | H | O | 1,3-cyclobutyl | OCH₂Ph |
| 1-731 | 0 | H | O | 1,3-cyclobutyl | NHSO₂Me |
| 1-732 | 0 | H | O | 1,3-cyclobutyl | NHSO₂N(Me)(CHMe₂) |
| 1-733 | 0 | F | O | 1,3-cyclobutyl | OH |
| 1-734 | 0 | F | O | 1,3-cyclobutyl | OMe |
| 1-735 | 0 | F | O | 1,3-cyclobutyl | OEt |
| 1-736 | 0 | F | O | 1,3-cyclobutyl | OCH₂Ph |
| 1-737 | 0 | F | O | 1,3-cyclobutyl | NHSO₂Me |
| 1-738 | 0 | F | O | 1,3-cyclobutyl | NHSO₂N(Me)(CHMe₂) |
| 1-739 | 0 | H | NH | 1,3-cyclobutyl | OH |
| 1-740 | 0 | H | NH | 1,3-cyclobutyl | OMe |
| 1-741 | 0 | H | NH | 1,3-cyclobutyl | OEt |
| 1-742 | 0 | H | NH | 1,3-cyclobutyl | OCH₂Ph |
| 1-743 | 0 | H | NH | 1,3-cyclobutyl | NHSO₂Me |
| 1-744 | 0 | H | NH | 1,3-cyclobutyl | NHSO₂N(Me)(CHMe₂) |
| 1-745 | 0 | F | NH | 1,3-cyclobutyl | OH |
| 1-746 | 0 | F | NH | 1,3-cyclobutyl | OMe |
| 1-747 | 0 | F | NH | 1,3-cyclobutyl | OEt |
| 1-748 | 0 | F | NH | 1,3-cyclobutyl | OCH₂Ph |
| 1-749 | 0 | F | NH | 1,3-cyclobutyl | NHSO₂Me |
| 1-750 | 0 | F | NH | 1,3-cyclobutyl | NHSO₂N(Me)(CHMe₂) |
| 1-751 | 0 | H | O | CH₂CH₂CH₂CH₂ | OH |
| 1-752 | 0 | H | O | CH₂CH₂CH₂CH₂ | OMe |
| 1-753 | 0 | H | O | CH₂CH₂CH₂CH₂ | OEt |
| 1-754 | 0 | H | O | CH₂CH₂CH₂CH₂ | OCH₂Ph |
| 1-755 | 0 | H | O | CH₂CH₂CH₂CH₂ | NHSO₂Me |
| 1-756 | 0 | H | O | CH₂CH₂CH₂CH₂ | NHSO₂N(Me)(CHMe₂) |
| 1-757 | 0 | F | O | CH₂CH₂CH₂CH₂ | OH |
| 1-758 | 0 | F | O | CH₂CH₂CH₂CH₂ | OMe |
| 1-759 | 0 | F | O | CH₂CH₂CH₂CH₂ | OEt |
| 1-760 | 0 | F | O | CH₂CH₂CH₂CH₂ | OCH₂Ph |
| 1-761 | 0 | F | O | CH₂CH₂CH₂CH₂ | NHSO₂Me |
| 1-762 | 0 | F | O | CH₂CH₂CH₂CH₂ | NHSO₂N(Me)(CHMe₂) |
| 1-763 | 0 | H | NH | CH₂CH₂CH₂CH₂ | OH |
| 1-764 | 0 | H | NH | CH₂CH₂CH₂CH₂ | OMe |
| 1-765 | 0 | H | NH | CH₂CH₂CH₂CH₂ | OEt |
| 1-766 | 0 | H | NH | CH₂CH₂CH₂CH₂ | OCH₂Ph |
| 1-767 | 0 | H | NH | CH₂CH₂CH₂CH₂ | NHSO₂Me |
| 1-768 | 0 | H | NH | CH₂CH₂CH₂CH₂ | NHSO₂N(Me)(CHMe₂) |
| 1-769 | 0 | F | NH | CH₂CH₂CH₂CH₂ | OH |
| 1-770 | 0 | F | NH | CH₂CH₂CH₂CH₂ | OMe |
| 1-771 | 0 | F | NH | CH₂CH₂CH₂CH₂ | OEt |
| 1-772 | 0 | F | NH | CH₂CH₂CH₂CH₂ | OCH₂Ph |
| 1-773 | 0 | F | NH | CH₂CH₂CH₂CH₂ | NHSO₂Me |
| 1-774 | 0 | F | NH | CH₂CH₂CH₂CH₂ | NHSO₂N(Me)(CHMe₂) |
| 1-775 | 0 | H | O | CHMeCH₂CH₂CH₂ | OH |
| 1-776 | 0 | H | O | CHMeCH₂CH₂CH₂ | OMe |
| 1-777 | 0 | H | O | CHMeCH₂CH₂CH₂ | OEt |
| 1-778 | 0 | H | O | CHMeCH₂CH₂CH₂ | OCH₂Ph |
| 1-779 | 0 | H | O | CHMeCH₂CH₂CH₂ | NHSO₂Me |
| 1-780 | 0 | H | O | CHMeCH₂CH₂CH₂ | NHSO₂N(Me)(CHMe₂) |
| 1-781 | 0 | F | O | CHMeCH₂CH₂CH₂ | OH |
| 1-782 | 0 | F | O | CHMeCH₂CH₂CH₂ | OMe |
| 1-783 | 0 | F | O | CHMeCH₂CH₂CH₂ | OEt |
| 1-784 | 0 | F | O | CHMeCH₂CH₂CH₂ | OCH₂Ph |
| 1-785 | 0 | F | O | CHMeCH₂CH₂CH₂ | NHSO₂Me |
| 1-786 | 0 | F | O | CHMeCH₂CH₂CH₂ | NHSO₂N(Me)(CHMe₂) |
| 1-787 | 0 | H | NH | CHMeCH₂CH₂CH₂ | OH |
| 1-788 | 0 | H | NH | CHMeCH₂CH₂CH₂ | OMe |
| 1-789 | 0 | H | NH | CHMeCH₂CH₂CH₂ | OEt |
| 1-790 | 0 | H | NH | CHMeCH₂CH₂CH₂ | OCH₂Ph |
| 1-791 | 0 | H | NH | CHMeCH₂CH₂CH₂ | NHSO₂Me |
| 1-792 | 0 | H | NH | CHMeCH₂CH₂CH₂ | NHSO₂N(Me)(CHMe₂) |
| 1-793 | 0 | F | NH | CHMeCH₂CH₂CH₂ | OH |
| 1-794 | 0 | F | NH | CHMeCH₂CH₂CH₂ | OMe |
| 1-795 | 0 | F | NH | CHMeCH₂CH₂CH₂ | OEt |
| 1-796 | 0 | F | NH | CHMeCH₂CH₂CH₂ | OCH₂Ph |
| 1-797 | 0 | F | NH | CHMeCH₂CH₂CH₂ | NHSO₂Me |
| 1-798 | 0 | F | NH | CHMeCH₂CH₂CH₂ | NHSO₂N(Me)(CHMe₂) |
| 1-799 | 1 | H | O | CH₂ | OH |
| 1-800 | 1 | H | O | CH₂ | OMe |
| 1-801 | 1 | H | O | CH₂ | OEt |
| 1-802 | 1 | H | O | CH₂ | OCH₂Ph |
| 1-803 | 1 | H | O | CH₂ | NHSO₂Me |
| 1-804 | 1 | H | O | CH₂ | NHSO₂N(Me)(CHMe₂) |
| 1-805 | 1 | F | O | CH₂ | OH |
| 1-806 | 1 | F | O | CH₂ | OMe |
| 1-807 | 1 | F | O | CH₂ | OEt |
| 1-808 | 1 | F | O | CH₂ | OCH₂Ph |
| 1-809 | 1 | F | O | CH₂ | NHSO₂Me |
| 1-810 | 1 | F | O | CH₂ | NHSO₂N(Me)(CHMe₂) |
| 1-811 | 1 | H | NH | CH₂ | OH |
| 1-812 | 1 | H | NH | CH₂ | OMe |
| 1-813 | 1 | H | NH | CH₂ | OEt |
| 1-814 | 1 | H | NH | CH₂ | OCH₂Ph |
| 1-815 | 1 | H | NH | CH₂ | NHSO₂Me |
| 1-816 | 1 | H | NH | CH₂ | NHSO₂N(Me)(CHMe₂) |
| 1-817 | 1 | F | NH | CH₂ | OH |
| 1-818 | 1 | F | NH | CH₂ | OMe |
| 1-819 | 1 | F | NH | CH₂ | OEt |
| 1-820 | 1 | F | NH | CH₂ | OCH₂Ph |
| 1-821 | 1 | F | NH | CH₂ | NHSO₂Me |
| 1-822 | 1 | F | NH | CH₂ | NHSO₂N(Me)(CHMe₂) |
| 1-823 | 1 | H | O | CHMe | OH |
| 1-824 | 1 | H | O | CHMe | OMe |
| 1-825 | 1 | H | O | CHMe | OEt |
| 1-826 | 1 | H | O | CHMe | OCH₂Ph |
| 1-827 | 1 | H | O | CHMe | NHSO₂Me |
| 1-828 | 1 | H | O | CHMe | NHSO₂N(Me)(CHMe₂) |
| 1-829 | 1 | F | O | CHMe | OH |
| 1-830 | 1 | F | O | CHMe | OMe |
| 1-831 | 1 | F | O | CHMe | OEt |
| 1-832 | 1 | F | O | CHMe | OCH₂Ph |
| 1-833 | 1 | F | O | CHMe | NHSO₂Me |
| 1-834 | 1 | F | O | CHMe | NHSO₂N(Me)(CHMe₂) |
| 1-835 | 1 | H | NH | CHMe | OH |
| 1-836 | 1 | H | NH | CHMe | OMe |
| 1-837 | 1 | H | NH | CHMe | OEt |
| 1-838 | 1 | H | NH | CHMe | OCH₂Ph |
| 1-839 | 1 | H | NH | CHMe | NHSO₂Me |
| 1-840 | 1 | H | NH | CHMe | NHSO₂N(Me)(CHMe₂) |
| 1-841 | 1 | F | NH | CHMe | OH |
| 1-842 | 1 | F | NH | CHMe | OMe |
| 1-843 | 1 | F | NH | CHMe | OEt |
| 1-844 | 1 | F | NH | CHMe | OCH₂Ph |
| 1-845 | 1 | F | NH | CHMe | NHSO₂Me |
| 1-846 | 1 | F | NH | CHMe | NHSO₂N(Me)(CHMe₂) |
| 1-847 | 1 | H | O | C(Me)₂ | OH |
| 1-848 | 1 | H | O | C(Me)₂ | OMe |
| 1-849 | 1 | H | O | C(Me)₂ | OEt |
| 1-850 | 1 | H | O | C(Me)₂ | OCH₂Ph |
| 1-851 | 1 | H | O | C(Me)₂ | NHSO₂Me |
| 1-852 | 1 | H | O | C(Me)₂ | NHSO₂N(Me)(CHMe₂) |
| 1-853 | 1 | F | O | C(Me)₂ | OH |
| 1-854 | 1 | F | O | C(Me)₂ | OMe |
| 1-855 | 1 | F | O | C(Me)₂ | OEt |
| 1-856 | 1 | F | O | C(Me)₂ | OCH₂Ph |
| 1-857 | 1 | F | O | C(Me)₂ | NHSO₂Me |
| 1-858 | 1 | F | O | C(Me)₂ | NHSO₂N(Me)(CHMe₂) |
| 1-859 | 1 | H | NH | C(Me)₂ | OH |
| 1-860 | 1 | H | NH | C(Me)₂ | OMe |
| 1-861 | 1 | H | NH | C(Me)₂ | OEt |
| 1-862 | 1 | H | NH | C(Me)₂ | OCH₂Ph |
| 1-863 | 1 | H | NH | C(Me)₂ | NHSO₂Me |
| 1-864 | 1 | H | NH | C(Me)₂ | NHSO₂N(Me)(CHMe₂) |
| 1-865 | 1 | F | NH | C(Me)₂ | OH |
| 1-866 | 1 | F | NH | C(Me)₂ | OMe |
| 1-867 | 1 | F | NH | C(Me)₂ | OEt |
| 1-868 | 1 | F | NH | C(Me)₂ | OCH₂Ph |
| 1-869 | 1 | F | NH | C(Me)₂ | NHSO₂Me |
| 1-870 | 1 | F | NH | C(Me)₂ | NHSO₂N(Me)(CHMe₂) |
| 1-871 | 1 | H | O | CH₂CH₂ | OH |
| 1-872 | 1 | H | O | CH₂CH₂ | OMe |
| 1-873 | 1 | H | O | CH₂CH₂ | OEt |
| 1-874 | 1 | H | O | CH₂CH₂ | OCH₂Ph |
| 1-875 | 1 | H | O | CH₂CH₂ | NHSO₂Me |
| 1-876 | 1 | H | O | CH₂CH₂ | NHSO₂N(Me)(CHMe₂) |
| 1-877 | 1 | F | O | CH₂CH₂ | OH |
| 1-878 | 1 | F | O | CH₂CH₂ | OMe |
| 1-879 | 1 | F | O | CH₂CH₂ | OEt |
| 1-880 | 1 | F | O | CH₂CH₂ | OCH₂Ph |
| 1-881 | 1 | F | O | CH₂CH₂ | NHSO₂Me |
| 1-882 | 1 | F | O | CH₂CH₂ | NHSO₂N(Me)(CHMe₂) |
| 1-883 | 1 | H | NH | CH₂CH₂ | OH |
| 1-884 | 1 | H | NH | CH₂CH₂ | OMe |
| 1-885 | 1 | H | NH | CH₂CH₂ | OEt |
| 1-886 | 1 | H | NH | CH₂CH₂ | OCH₂Ph |
| 1-887 | 1 | H | NH | CH₂CH₂ | NHSO₂Me |
| 1-888 | 1 | H | NH | CH₂CH₂ | NHSO₂N(Me)(CHMe₂) |
| 1-889 | 1 | F | NH | CH₂CH₂ | OH |
| 1-890 | 1 | F | NH | CH₂CH₂ | OMe |
| 1-891 | 1 | F | NH | CH₂CH₂ | OEt |
| 1-892 | 1 | F | NH | CH₂CH₂ | OCH₂Ph |
| 1-893 | 1 | F | NH | CH₂CH₂ | NHSO₂Me |
| 1-894 | 1 | F | NH | CH₂CH₂ | NHSO₂N(Me)(CHMe₂) |
| 1-895 | 1 | H | O | CHMeCH₂ | OH |
| 1-896 | 1 | H | O | CHMeCH₂ | OMe |
| 1-897 | 1 | H | O | CHMeCH₂ | OEt |
| 1-898 | 1 | H | O | CHMeCH₂ | OCH₂Ph |
| 1-899 | 1 | H | O | CHMeCH₂ | NHSO₂Me |
| 1-900 | 1 | H | O | CHMeCH₂ | NHSO₂N(Me)(CHMe₂) |
| 1-901 | 1 | F | O | CHMeCH₂ | OH |
| 1-902 | 1 | F | O | CHMeCH₂ | OMe |
| 1-903 | 1 | F | O | CHMeCH₂ | OEt |
| 1-904 | 1 | F | O | CHMeCH₂ | OCH₂Ph |
| 1-905 | 1 | F | O | CHMeCH₂ | NHSO₂Me |
| 1-906 | 1 | F | O | CHMeCH₂ | NHSO₂N(Me)(CHMe₂) |
| 1-907 | 1 | H | NH | CHMeCH₂ | OH |
| 1-908 | 1 | H | NH | CHMeCH₂ | OMe |
| 1-909 | 1 | H | NH | CHMeCH₂ | OEt |
| 1-910 | 1 | H | NH | CHMeCH₂ | OCH₂Ph |
| 1-911 | 1 | H | NH | CHMeCH₂ | NHSO₂Me |
| 1-912 | 1 | H | NH | CHMeCH₂ | NHSO₂N(Me)(CHMe₂) |
| 1-913 | 1 | F | NH | CHMeCH₂ | OH |
| 1-914 | 1 | F | NH | CHMeCH₂ | OMe |
| 1-915 | 1 | F | NH | CHMeCH₂ | OEt |
| 1-916 | 1 | F | NH | CHMeCH₂ | OCH₂Ph |
| 1-917 | 1 | F | NH | CHMeCH₂ | NHSO₂Me |
| 1-918 | 1 | F | NH | CHMeCH₂ | NHSO₂N(Me)(CHMe₂) |
| 1-919 | 1 | H | O | CH₂CHMe | OH |
| 1-920 | 1 | H | O | CH₂CHMe | OMe |
| 1-921 | 1 | H | O | CH₂CHMe | OEt |
| 1-922 | 1 | H | O | CH₂CHMe | OCH₂Ph |
| 1-923 | 1 | H | O | CH₂CHMe | NHSO₂Me |
| 1-924 | 1 | H | O | CH₂CHMe | NHSO₂N(Me)(CHMe₂) |
| 1-925 | 1 | F | O | CH₂CHMe | OH |
| 1-926 | 1 | F | O | CH₂CHMe | OMe |
| 1-927 | 1 | F | O | CH₂CHMe | OEt |
| 1-928 | 1 | F | O | CH₂CHMe | OCH₂Ph |
| 1-929 | 1 | F | O | CH₂CHMe | NHSO₂Me |
| 1-930 | 1 | F | O | CH₂CHMe | NHSO₂N(Me)(CHMe₂) |
| 1-931 | 1 | H | NH | CH₂CHMe | OH |
| 1-932 | 1 | H | NH | CH₂CHMe | OMe |
| 1-933 | 1 | H | NH | CH₂CHMe | OEt |
| 1-934 | 1 | H | NH | CH₂CHMe | OCH₂Ph |
| 1-935 | 1 | H | NH | CH₂CHMe | NHSO₂Me |
| 1-936 | 1 | H | NH | CH₂CHMe | NHSO₂N(Me)(CHMe₂) |
| 1-937 | 1 | F | NH | CH₂CHMe | OH |
| 1-938 | 1 | F | NH | CH₂CHMe | OMe |
| 1-939 | 1 | F | NH | CH₂CHMe | OEt |
| 1-940 | 1 | F | NH | CH₂CHMe | OCH₂Ph |
| 1-941 | 1 | F | NH | CH₂CHMe | NHSO₂Me |
| 1-942 | 1 | F | NH | CH₂CHMe | NHSO₂N(Me)(CHMe₂) |
| 1-943 | 1 | H | O | CMe₂CH₂ | OH |
| 1-944 | 1 | H | O | CMe₂CH₂ | OMe |
| 1-945 | 1 | H | O | CMe₂CH₂ | OEt |
| 1-946 | 1 | H | O | CMe₂CH₂ | OCH₂Ph |
| 1-947 | 1 | H | O | CMe₂CH₂ | NHSO₂Me |
| 1-948 | 1 | H | O | CMe₂CH₂ | NHSO₂N(Me)(CHMe₂) |
| 1-949 | 1 | F | O | CMe₂CH₂ | OH |
| 1-950 | 1 | F | O | CMe₂CH₂ | OMe |
| 1-951 | 1 | F | O | CMe₂CH₂ | OEt |
| 1-952 | 1 | F | O | CMe₂CH₂ | OCH₂Ph |
| 1-953 | 1 | F | O | CMe₂CH₂ | NHSO₂Me |
| 1-954 | 1 | F | O | CMe₂CH₂ | NHSO₂N(Me)(CHMe₂) |
| 1-955 | 1 | H | NH | CMe₂CH₂ | OH |
| 1-956 | 1 | H | NH | CMe₂CH₂ | OMe |
| 1-957 | 1 | H | NH | CMe₂CH₂ | OEt |
| 1-958 | 1 | H | NH | CMe₂CH₂ | OCH₂Ph |
| 1-959 | 1 | H | NH | CMe₂CH₂ | NHSO₂Me |
| 1-960 | 1 | H | NH | CMe₂CH₂ | NHSO₂N(Me)(CHMe₂) |
| 1-961 | 1 | F | NH | CMe₂CH₂ | OH |
| 1-962 | 1 | F | NH | CMe₂CH₂ | OMe |
| 1-963 | 1 | F | NH | CMe₂CH₂ | OEt |
| 1-964 | 1 | F | NH | CMe₂CH₂ | OCH₂Ph |
| 1-965 | 1 | F | NH | CMe₂CH₂ | NHSO₂Me |
| 1-966 | 1 | F | NH | CMe₂CH₂ | NHSO₂N(Me)(CHMe₂) |
| 1-967 | 1 | H | O | CH₂CMe₂ | OH |
| 1-968 | 1 | H | O | CH₂CMe₂ | OMe |
| 1-969 | 1 | H | O | CH₂CMe₂ | OEt |
| 1-970 | 1 | H | O | CH₂CMe₂ | OCH₂Ph |
| 1-971 | 1 | H | O | CH₂CMe₂ | NHSO₂Me |
| 1-972 | 1 | H | O | CH₂CMe₂ | NHSO₂N(Me)(CHMe₂) |
| 1-973 | 1 | F | O | CH₂CMe₂ | OH |
| 1-974 | 1 | F | O | CH₂CMe₂ | OMe |
| 1-975 | 1 | F | O | CH₂CMe₂ | OEt |
| 1-976 | 1 | F | O | CH₂CMe₂ | OCH₂Ph |
| 1-977 | 1 | F | O | CH₂CMe₂ | NHSO₂Me |
| 1-978 | 1 | F | O | CH₂CMe₂ | NHSO₂N(Me)(CHMe₂) |
| 1-979 | 1 | H | NH | CH₂CMe₂ | OH |
| 1-980 | 1 | H | NH | CH₂CMe₂ | OMe |
| 1-981 | 1 | H | NH | CH₂CMe₂ | OEt |
| 1-982 | 1 | H | NH | CH₂CMe₂ | OCH₂Ph |
| 1-983 | 1 | H | NH | CH₂CMe₂ | NHSO₂Me |
| 1-984 | 1 | H | NH | CH₂CMe₂ | NHSO₂N(Me)(CHMe₂) |
| 1-985 | 1 | F | NH | CH₂CMe₂ | OH |
| 1-986 | 1 | F | NH | CH₂CMe₂ | OMe |
| 1-987 | 1 | F | NH | CH₂CMe₂ | OEt |
| 1-988 | 1 | F | NH | CH₂CMe₂ | OCH₂Ph |
| 1-989 | 1 | F | NH | CH₂CMe₂ | NHSO₂Me |
| 1-990 | 1 | F | NH | CH₂CMe₂ | NHSO₂N(Me)(CHMe₂) |
| 1-991 | 1 | H | O | CH₂CH₂CH₂ | OH |
| 1-992 | 1 | H | O | CH₂CH₂CH₂ | OMe |
| 1-993 | 1 | H | O | CH₂CH₂CH₂ | OEt |
| 1-994 | 1 | H | O | CH₂CH₂CH₂ | OCH₂Ph |
| 1-995 | 1 | H | O | CH₂CH₂CH₂ | NHSO₂Me |
| 1-996 | 1 | H | O | CH₂CH₂CH₂ | NHSO₂N(Me)(CHMe₂) |
| 1-997 | 1 | F | O | CH₂CH₂CH₂ | OH |
| 1-998 | 1 | F | O | CH₂CH₂CH₂ | OMe |
| 1-999 | 1 | F | O | CH₂CH₂CH₂ | OEt |
| 1-1000 | 1 | F | O | CH₂CH₂CH₂ | OCH₂Ph |
| 1-1001 | 1 | F | O | CH₂CH₂CH₂ | NHSO₂Me |
| 1-1002 | 1 | F | O | CH₂CH₂CH₂ | NHSO₂N(Me)(CHMe₂) |
| 1-1003 | 1 | H | NH | CH₂CH₂CH₂ | OH |
| 1-1004 | 1 | H | NH | CH₂CH₂CH₂ | OMe |
| 1-1005 | 1 | H | NH | CH₂CH₂CH₂ | OEt |
| 1-1006 | 1 | H | NH | CH₂CH₂CH₂ | OCH₂Ph |
| 1-1007 | 1 | H | NH | CH₂CH₂CH₂ | NHSO₂Me |
| 1-1008 | 1 | H | NH | CH₂CH₂CH₂ | NHSO₂N(Me)(CHMe₂) |
| 1-1009 | 1 | F | NH | CH₂CH₂CH₂ | OH |
| 1-1010 | 1 | F | NH | CH₂CH₂CH₂ | OMe |
| 1-1011 | 1 | F | NH | CH₂CH₂CH₂ | OEt |
| 1-1012 | 1 | F | NH | CH₂CH₂CH₂ | OCH₂Ph |
| 1-1013 | 1 | F | NH | CH₂CH₂CH₂ | NHSO₂Me |
| 1-1014 | 1 | F | NH | CH₂CH₂CH₂ | NHSO₂N(Me)(CHMe₂) |
| 1-1015 | 2 | H | O | CH₂ | OH |
| 1-1016 | 2 | H | O | CH₂ | OMe |
| 1-1017 | 2 | H | O | CH₂ | OEt |
| 1-1018 | 2 | H | O | CH₂ | OCH₂Ph |
| 1-1019 | 2 | H | O | CH₂ | NHSO₂Me |
| 1-1020 | 2 | H | O | CH₂ | NHSO₂N(Me)(CHMe₂) |
| 1-1021 | 2 | F | O | CH₂ | OH |
| 1-1022 | 2 | F | O | CH₂ | OMe |
| 1-1023 | 2 | F | O | CH₂ | OEt |
| 1-1024 | 2 | F | O | CH₂ | OCH₂Ph |
| 1-1025 | 2 | F | O | CH₂ | NHSO₂Me |
| 1-1026 | 2 | F | O | CH₂ | NHSO₂N(Me)(CHMe₂) |
| 1-1027 | 2 | H | NH | CH₂ | OH |
| 1-1028 | 2 | H | NH | CH₂ | OMe |
| 1-1029 | 2 | H | NH | CH₂ | OEt |
| 1-1030 | 2 | H | NH | CH₂ | OCH₂Ph |
| 1-1031 | 2 | H | NH | CH₂ | NHSO₂Me |
| 1-1032 | 2 | H | NH | CH₂ | NHSO₂N(Me)(CHMe₂) |
| 1-1033 | 2 | F | NH | CH₂ | OH |
| 1-1034 | 2 | F | NH | CH₂ | OMe |
| 1-1035 | 2 | F | NH | CH₂ | OEt |
| 1-1036 | 2 | F | NH | CH₂ | OCH₂Ph |
| 1-1037 | 2 | F | NH | CH₂ | NHSO₂Me |
| 1-1038 | 2 | F | NH | CH₂ | NHSO₂N(Me)(CHMe₂) |
| 1-1039 | 2 | H | O | CHMe | OH |
| 1-1040 | 2 | H | O | CHMe | OMe |
| 1-1041 | 2 | H | O | CHMe | OEt |
| 1-1042 | 2 | H | O | CHMe | OCH₂Ph |
| 1-1043 | 2 | H | O | CHMe | NHSO₂Me |
| 1-1044 | 2 | H | O | CHMe | NHSO₂N(Me)(CHMe₂) |
| 1-1045 | 2 | F | O | CHMe | OH |
| 1-1046 | 2 | F | O | CHMe | OMe |
| 1-1047 | 2 | F | O | CHMe | OEt |
| 1-1048 | 2 | F | O | CHMe | OCH₂Ph |
| 1-1049 | 2 | F | O | CHMe | NHSO₂Me |
| 1-1050 | 2 | F | O | CHMe | NHSO₂N(Me)(CHMe₂) |
| 1-1051 | 2 | H | NH | CHMe | OH |
| 1-1052 | 2 | H | NH | CHMe | OMe |
| 1-1053 | 2 | H | NH | CHMe | OEt |
| 1-1054 | 2 | H | NH | CHMe | OCH₂Ph |
| 1-1055 | 2 | H | NH | CHMe | NHSO₂Me |
| 1-1056 | 2 | H | NH | CHMe | NHSO₂N(Me)(CHMe₂) |
| 1-1057 | 2 | F | NH | CHMe | OH |
| 1-1058 | 2 | F | NH | CHMe | OMe |
| 1-1059 | 2 | F | NH | CHMe | OEt |
| 1-1060 | 2 | F | NH | CHMe | OCH₂Ph |
| 1-1061 | 2 | F | NH | CHMe | NHSO₂Me |
| 1-1062 | 2 | F | NH | CHMe | NHSO₂N(Me)(CHMe₂) |
| 1-1063 | 2 | H | O | C(Me)₂ | OH |
| 1-1064 | 2 | H | O | C(Me)₂ | OMe |
| 1-1065 | 2 | H | O | C(Me)₂ | OEt |
| 1-1066 | 2 | H | O | C(Me)₂ | OCH₂Ph |
| 1-1067 | 2 | H | O | C(Me)₂ | NHSO₂Me |
| 1-1068 | 2 | H | O | C(Me)₂ | NHSO₂N(Me)(CHMe₂) |
| 1-1069 | 2 | F | O | C(Me)₂ | OH |
| 1-1070 | 2 | F | O | C(Me)₂ | OMe |
| 1-1071 | 2 | F | O | C(Me)₂ | OEt |
| 1-1072 | 2 | F | O | C(Me)₂ | OCH₂Ph |
| 1-1073 | 2 | F | O | C(Me)₂ | NHSO₂Me |
| 1-1074 | 2 | F | O | C(Me)₂ | NHSO₂N(Me)(CHMe₂) |
| 1-1075 | 2 | H | NH | C(Me)₂ | OH |
| 1-1076 | 2 | H | NH | C(Me)₂ | OMe |
| 1-1077 | 2 | H | NH | C(Me)₂ | OEt |
| 1-1078 | 2 | H | NH | C(Me)₂ | OCH₂Ph |
| 1-1079 | 2 | H | NH | C(Me)₂ | NHSO₂Me |
| 1-1080 | 2 | H | NH | C(Me)₂ | NHSO₂N(Me)(CHMe₂) |
| 1-1081 | 2 | F | NH | C(Me)₂ | OH |
| 1-1082 | 2 | F | NH | C(Me)₂ | OMe |
| 1-1083 | 2 | F | NH | C(Me)₂ | OEt |
| 1-1084 | 2 | F | NH | C(Me)₂ | OCH₂Ph |
| 1-1085 | 2 | F | NH | C(Me)₂ | NHSO₂Me |
| 1-1086 | 2 | F | NH | C(Me)₂ | NHSO₂N(Me)(CHMe₂) |
| 1-1087 | 2 | H | O | CH₂CH₂ | OH |
| 1-1088 | 2 | H | O | CH₂CH₂ | OMe |
| 1-1089 | 2 | H | O | CH₂CH₂ | OEt |
| 1-1090 | 2 | H | O | CH₂CH₂ | OCH₂Ph |
| 1-1091 | 2 | H | O | CH₂CH₂ | NHSO₂Me |
| 1-1092 | 2 | H | O | CH₂CH₂ | NHSO₂N(Me)(CHMe₂) |
| 1-1093 | 2 | F | O | CH₂CH₂ | OH |
| 1-1094 | 2 | F | O | CH₂CH₂ | OMe |
| 1-1095 | 2 | F | O | CH₂CH₂ | OEt |
| 1-1096 | 2 | F | O | CH₂CH₂ | OCH₂Ph |
| 1-1097 | 2 | F | O | CH₂CH₂ | NHSO₂Me |
| 1-1098 | 2 | F | O | CH₂CH₂ | NHSO₂N(Me)(CHMe₂) |
| 1-1099 | 2 | H | NH | CH₂CH₂ | OH |
| 1-1100 | 2 | H | NH | CH₂CH₂ | OMe |
| 1-1101 | 2 | H | NH | CH₂CH₂ | OEt |
| 1-1102 | 2 | H | NH | CH₂CH₂ | OCH₂Ph |
| 1-1103 | 2 | H | NH | CH₂CH₂ | NHSO₂Me |
| 1-1104 | 2 | H | NH | CH₂CH₂ | NHSO₂N(Me)(CHMe₂) |
| 1-1105 | 2 | F | NH | CH₂CH₂ | OH |
| 1-1106 | 2 | F | NH | CH₂CH₂ | OMe |
| 1-1107 | 2 | F | NH | CH₂CH₂ | OEt |
| 1-1108 | 2 | F | NH | CH₂CH₂ | OCH₂Ph |
| 1-1109 | 2 | F | NH | CH₂CH₂ | NHSO₂Me |
| 1-1110 | 2 | F | NH | CH₂CH₂ | NHSO₂N(Me)(CHMe₂) |
| 1-1111 | 2 | H | O | CHMeCH₂ | OH |
| 1-1112 | 2 | H | O | CHMeCH₂ | OMe |
| 1-1113 | 2 | H | O | CHMeCH₂ | OEt |
| 1-1114 | 2 | H | O | CHMeCH₂ | OCH₂Ph |
| 1-1115 | 2 | H | O | CHMeCH₂ | NHSO₂Me |
| 1-1116 | 2 | H | O | CHMeCH₂ | NHSO₂N(Me)(CHMe₂) |
| 1-1117 | 2 | F | O | CHMeCH₂ | OH |
| 1-1118 | 2 | F | O | CHMeCH₂ | OMe |
| 1-1119 | 2 | F | O | CHMeCH₂ | OEt |
| 1-1120 | 2 | F | O | CHMeCH₂ | OCH₂Ph |
| 1-1121 | 2 | F | O | CHMeCH₂ | NHSO₂Me |
| 1-1122 | 2 | F | O | CHMeCH₂ | NHSO₂N(Me)(CHMe₂) |
| 1-1123 | 2 | H | NH | CHMeCH₂ | OH |
| 1-1124 | 2 | H | NH | CHMeCH₂ | OMe |
| 1-1125 | 2 | H | NH | CHMeCH₂ | OEt |
| 1-1126 | 2 | H | NH | CHMeCH₂ | OCH₂Ph |
| 1-1127 | 2 | H | NH | CHMeCH₂ | NHSO₂Me |
| 1-1128 | 2 | H | NH | CHMeCH₂ | NHSO₂N(Me)(CHMe₂) |
| 1-1129 | 2 | F | NH | CHMeCH₂ | OH |
| 1-1130 | 2 | F | NH | CHMeCH₂ | OMe |
| 1-1131 | 2 | F | NH | CHMeCH₂ | OEt |
| 1-1132 | 2 | F | NH | CHMeCH₂ | OCH₂Ph |
| 1-1133 | 2 | F | NH | CHMeCH₂ | NHSO₂Me |
| 1-1134 | 2 | F | NH | CHMeCH₂ | NHSO₂N(Me)(CHMe₂) |
| 1-1135 | 2 | H | O | CH₂CHMe | OH |
| 1-1136 | 2 | H | O | CH₂CHMe | OMe |
| 1-1137 | 2 | H | O | CH₂CHMe | OEt |
| 1-1138 | 2 | H | O | CH₂CHMe | OCH₂Ph |
| 1-1139 | 2 | H | O | CH₂CHMe | NHSO₂Me |
| 1-1140 | 2 | H | O | CH₂CHMe | NHSO₂N(Me)(CHMe₂) |
| 1-1141 | 2 | F | O | CH₂CHMe | OH |
| 1-1142 | 2 | F | O | CH₂CHMe | OMe |
| 1-1143 | 2 | F | O | CH₂CHMe | OEt |
| 1-1144 | 2 | F | O | CH₂CHMe | OCH₂Ph |
| 1-1145 | 2 | F | O | CH₂CHMe | NHSO₂Me |
| 1-1146 | 2 | F | O | CH₂CHMe | NHSO₂N(Me)(CHMe₂) |
| 1-1147 | 2 | H | NH | CH₂CHMe | OH |
| 1-1148 | 2 | H | NH | CH₂CHMe | OMe |
| 1-1149 | 2 | H | NH | CH₂CHMe | OEt |
| 1-1150 | 2 | H | NH | CH₂CHMe | OCH₂Ph |
| 1-1151 | 2 | H | NH | CH₂CHMe | NHSO₂Me |
| 1-1152 | 2 | H | NH | CH₂CHMe | NHSO₂N(Me)(CHMe₂) |
| 1-1153 | 2 | F | NH | CH₂CHMe | OH |
| 1-1154 | 2 | F | NH | CH₂CHMe | OMe |
| 1-1155 | 2 | F | NH | CH₂CHMe | OEt |
| 1-1156 | 2 | F | NH | CH₂CHMe | OCH₂Ph |
| 1-1157 | 2 | F | NH | CH₂CHMe | NHSO₂Me |
| 1-1158 | 2 | F | NH | CH₂CHMe | NHSO₂N(Me)(CHMe₂) |
| 1-1159 | 2 | H | O | CMe₂CH₂ | OH |
| 1-1160 | 2 | H | O | CMe₂CH₂ | OMe |
| 1-1161 | 2 | H | O | CMe₂CH₂ | OEt |
| 1-1162 | 2 | H | O | CMe₂CH₂ | OCH₂Ph |
| 1-1163 | 2 | H | O | CMe₂CH₂ | NHSO₂Me |
| 1-1164 | 2 | H | O | CMe₂CH₂ | NHSO₂N(Me)(CHMe₂) |
| 1-1165 | 2 | F | O | CMe₂CH₂ | OH |
| 1-1166 | 2 | F | O | CMe₂CH₂ | OMe |
| 1-1167 | 2 | F | O | CMe₂CH₂ | OEt |
| 1-1168 | 2 | F | O | CMe₂CH₂ | OCH₂Ph |
| 1-1169 | 2 | F | O | CMe₂CH₂ | NHSO₂Me |
| 1-1170 | 2 | F | O | CMe₂CH₂ | NHSO₂N(Me)(CHMe₂) |
| 1-1171 | 2 | H | NH | CMe₂CH₂ | OH |
| 1-1172 | 2 | H | NH | CMe₂CH₂ | OMe |
| 1-1173 | 2 | H | NH | CMe₂CH₂ | OEt |
| 1-1174 | 2 | H | NH | CMe₂CH₂ | OCH₂Ph |
| 1-1175 | 2 | H | NH | CMe₂CH₂ | NHSO₂Me |
| 1-1176 | 2 | H | NH | CMe₂CH₂ | NHSO₂N(Me)(CHMe₂) |
| 1-1177 | 2 | F | NH | CMe₂CH₂ | OH |
| 1-1178 | 2 | F | NH | CMe₂CH₂ | OMe |
| 1-1179 | 2 | F | NH | CMe₂CH₂ | OEt |
| 1-1180 | 2 | F | NH | CMe₂CH₂ | OCH₂Ph |
| 1-1181 | 2 | F | NH | CMe₂CH₂ | NHSO₂Me |
| 1-1182 | 2 | F | NH | CMe₂CH₂ | NHSO₂N(Me)(CHMe₂) |
| 1-1183 | 2 | H | O | CH₂CMe₂ | OH |
| 1-1184 | 2 | H | O | CH₂CMe₂ | OMe |
| 1-1185 | 2 | H | O | CH₂CMe₂ | OEt |
| 1-1186 | 2 | H | O | CH₂CMe₂ | OCH₂Ph |
| 1-1187 | 2 | H | O | CH₂CMe₂ | NHSO₂Me |
| 1-1188 | 2 | H | O | CH₂CMe₂ | NHSO₂N(Me)(CHMe₂) |
| 1-1189 | 2 | F | O | CH₂CMe₂ | OH |
| 1-1190 | 2 | F | O | CH₂CMe₂ | OMe |
| 1-1191 | 2 | F | O | CH₂CMe₂ | OEt |
| 1-1192 | 2 | F | O | CH₂CMe₂ | OCH₂Ph |
| 1-1193 | 2 | F | O | CH₂CMe₂ | NHSO₂Me |
| 1-1194 | 2 | F | O | CH₂CMe₂ | NHSO₂N(Me)(CHMe₂) |
| 1-1195 | 2 | H | NH | CH₂CMe₂ | OH |
| 1-1196 | 2 | H | NH | CH₂CMe₂ | OMe |
| 1-1197 | 2 | H | NH | CH₂CMe₂ | OEt |
| 1-1198 | 2 | H | NH | CH₂CMe₂ | OCH₂Ph |
| 1-1199 | 2 | H | NH | CH₂CMe₂ | NHSO₂Me |
| 1-1200 | 2 | H | NH | CH₂CMe₂ | NHSO₂N(Me)(CHMe₂) |
| 1-1201 | 2 | F | NH | CH₂CMe₂ | OH |
| 1-1202 | 2 | F | NH | CH₂CMe₂ | OMe |
| 1-1203 | 2 | F | NH | CH₂CMe₂ | OEt |
| 1-1204 | 2 | F | NH | CH₂CMe₂ | OCH₂Ph |
| 1-1205 | 2 | F | NH | CH₂CMe₂ | NHSO₂Me |
| 1-1206 | 2 | F | NH | CH₂CMe₂ | NHSO₂N(Me)(CHMe₂) |
| 1-1207 | 2 | H | O | CH₂CH₂CH₂ | OH |
| 1-1208 | 2 | H | O | CH₂CH₂CH₂ | OMe |
| 1-1209 | 2 | H | O | CH₂CH₂CH₂ | OEt |
| 1-1210 | 2 | H | O | CH₂CH₂CH₂ | OCH₂Ph |
| 1-1211 | 2 | H | O | CH₂CH₂CH₂ | NHSO₂Me |
| 1-1212 | 2 | H | O | CH₂CH₂CH₂ | NHSO₂N(Me)(CHMe₂) |
| 1-1213 | 2 | F | O | CH₂CH₂CH₂ | OH |
| 1-1214 | 2 | F | O | CH₂CH₂CH₂ | OMe |
| 1-1215 | 2 | F | O | CH₂CH₂CH₂ | OEt |
| 1-1216 | 2 | F | O | CH₂CH₂CH₂ | OCH₂Ph |
| 1-1217 | 2 | F | O | CH₂CH₂CH₂ | NHSO₂Me |
| 1-1218 | 2 | F | O | CH₂CH₂CH₂ | NHSO₂N(Me)(CHMe₂) |
| 1-1219 | 2 | H | NH | CH₂CH₂CH₂ | OH |
| 1-1220 | 2 | H | NH | CH₂CH₂CH₂ | OMe |
| 1-1221 | 2 | H | NH | CH₂CH₂CH₂ | OEt |
| 1-1222 | 2 | H | NH | CH₂CH₂CH₂ | OCH₂Ph |
| 1-1223 | 2 | H | NH | CH₂CH₂CH₂ | NHSO₂Me |
| 1-1224 | 2 | H | NH | CH₂CH₂CH₂ | NHSO₂N(Me)(CHMe₂) |
| 1-1225 | 2 | F | NH | CH₂CH₂CH₂ | OH |
| 1-1226 | 2 | F | NH | CH₂CH₂CH₂ | OMe |
| 1-1227 | 2 | F | NH | CH₂CH₂CH₂ | OEt |
| 1-1228 | 2 | F | NH | CH₂CH₂CH₂ | OCH₂Ph |
| 1-1229 | 2 | F | NH | CH₂CH₂CH₂ | NHSO₂Me |
| 1-1230 | 2 | F | NH | CH₂CH₂CH₂ | NHSO₂N(Me)(CHMe₂) |

1230 compounds of formula (I), wherein X¹ and X² are sulfur, X³ is oxygen, R¹ and R² are methyl, R⁴ is chloro, R¹⁶ and R¹⁷ are hydrogen, and Y is C—H, and the values of m, B, D, R³ and R⁸ are as given in Table 1 for compounds 1-1 to 1-1230, are designated as compound numbers 2-1 to 2-1230 respectively.
1230 compounds of formula (I), wherein X¹ is sulfur, X² and X³ are oxygen, R¹ and R² are methyl, R⁴ is chloro, R¹⁶ and R¹⁷ are hydrogen, and Y is N, and the values of m, B, D, R³ and R⁸ are as given in Table 1 for compounds 1-1 to 1-1230, are designated as compound numbers 3-1 to 3-1230 respectively.
1230 compounds of formula (I), wherein X¹ and X² are sulfur, X³ is oxygen, R¹ and R² are methyl, R⁴ is chloro, R¹⁶ and R¹⁷ are hydrogen, and Y is N, and the values of m, B, D, R³ and R⁸ are as given in Table 1 for compounds 1-1 to 1-1230, are designated as compound numbers 4-1 to 4-1230 respectively.
1230 compounds of formula (I), wherein X¹ is sulfur, X² and X³ are oxygen, R¹ and R² are methyl, R⁴ is CN, R¹⁶ and R¹⁷ are hydrogen, and Y is N, and the values of m, B, D, R³ and R⁸ are as given in Table 1 for compounds 1-1 to 1-1230, are designated as compound numbers 5-1 to 5-1230 respectively.
1230 compounds of formula (I), wherein X¹ and X² are sulfur, X³ is oxygen, R¹ and R² are methyl, R⁴ is CN, R¹⁶ and R¹⁷ are hydrogen, and Y is C—H, and the values of m, B, D, R³ and R⁸ are as given in Table 1 for compounds 1-1 to 1-1230, are designated as compound numbers 6-1 to 6-1230 respectively.
1230 compounds of formula (I), wherein X¹ is sulfur, X² and X³ are oxygen, R¹ and R² are methyl, R⁴ is bromo, R¹⁶ and R¹⁷ are hydrogen, and Y is N, and the values of m, B, D, R³ and R⁸ are as given in Table 1 for compounds 1-1 to 1-1230, are designated as compound numbers 7-1 to 7-1230 respectively.
1230 compounds of formula (I), wherein X¹ and X² are sulfur, X³ is oxygen, R¹ and R² are methyl, R⁴ is bromo, R¹⁶ and R¹⁷ are hydrogen, and Y is C—H, and the values of m, B, D, R³ and R⁸ are as given in Table 1 for compounds 1-1 to 1-1230, are designated as compound numbers 8-1 to 8-1230 respectively.
1230 compounds of formula (I), wherein X¹ is sulfur, X² and X³ are oxygen, R¹ and R² are methyl, R⁴ is chloro, R¹⁶ is hydrogen, R¹⁷ is methyl and Y is C—H and the values of m, B, D, R³ and R⁸ are as given in Table 1 for compounds 1-1 to 1-1230, are designated as compound numbers 9-1 to 9-1230 respectively.
1230 compounds of formula (I), wherein X¹ and X² are sulfur, X³ is oxygen, R¹ and R² are methyl, R⁴ is chloro, R¹⁶ is hydrogen, R¹⁷ is methyl and Y is C—H, and the values of m, B, D, R³ and R⁸ are as given in Table 1 for compounds 1-1 to 1-1230, are designated as compound numbers 10-1 to 10-1230 respectively.
1230 compounds of formula (I), wherein X¹ is sulfur, X² and X³ are oxygen, R¹ and R² are methyl, R⁴ is chloro, R¹⁶ is hydrogen, R¹⁷ is methyl and Y is N, and the values of m, B, D, R³ and R⁸ are as given in Table 1 for compounds 1-1 to 1-1230, are designated as compound numbers 11-1 to 11-1230 respectively.
1230 compounds of formula (I), wherein X¹ and X² are sulfur, X³ is oxygen, R¹ and R² are methyl, R⁴ is chloro, R¹⁶ is hydrogen, R¹⁷ is methyl and Y is N, and the values of m, B, D, R³ and R⁸ are as given in Table 1 for compounds 1-1 to 1-1230, are designated as compound numbers 12-1 to 12-1230 respectively.
1230 compounds of formula (I), wherein X¹ is sulfur, X² and X³ are oxygen, R¹ and R² are methyl, R⁴ is CN, R¹⁶ is hydrogen, R¹⁷ is methyl and Y is N, and the values of m, B, D, R³ and R⁸ are as given in Table 1 for compounds 1-1 to 1-1230, are designated as compound numbers 13-1 to 13-1230 respectively.
1230 compounds of formula (I), wherein X¹ and X² are sulfur, X³ is oxygen, R¹ and R² are methyl, R⁴ is CN, R¹⁶ is hydrogen, R¹⁷ is methyl and Y is C—H, and the values of m, B, D, R³ and R⁸ are as given in Table 1 for compounds 1-1 to 1-1230, are designated as compound numbers 14-1 to 14-1230 respectively.
1230 compounds of formula (I), wherein X¹ is sulfur, X² and X³ are oxygen, R¹ and R² are methyl, R⁴ is bromo, R¹⁶ is hydrogen, R¹⁷ is methyl and Y is N, and the values of m, B, D, R³ and R⁸ are as given in Table 1 for compounds 1-1 to 1-1230, are designated as compound numbers 15-1 to 15-1230 respectively.
1230 compounds of formula (I), wherein X¹ and X² are sulfur, X³ is oxygen, R¹ and R² are methyl, R⁴ is bromo, R¹⁶ is hydrogen, R¹⁷ is methyl and Y is C—H, and the values of m, B, D, R³ and R⁸ are as given in Table 1 for compounds 1-1 to 1-1230, are designated as compound numbers 16-1 to 16-1230 respectively.
1230 compounds of formula (I), wherein X¹ is sulfur, X² and X³ are oxygen, R¹ and R² are methyl, R⁴ is chloro, R¹⁶ and R¹⁷ are methyl and Y is C—H and the values of m, B, D, R³ and R⁸ are as given in Table 1 for compounds 1-1 to 1-1230, are designated as compound numbers 17-1 to 17-1230 respectively.
1230 compounds of formula (I), wherein X¹ and X² are sulfur, X³ is oxygen, R¹ and R² are methyl, R⁴ is chloro, R¹⁶ and R¹⁷ are methyl, and Y is C—H, and the values of m, B, D, R³ and R⁸ are as given in Table 1 for compounds 1-1 to 1-1230, are designated as compound numbers 18-1 to 18-1230 respectively.
1230 compounds of formula (I), wherein X¹ is sulfur, X² and X³ are oxygen, R¹ and R² are methyl, R⁴ is chloro, R¹⁶ and R¹⁷ are methyl, and Y is N, and the values of m, B, D, R³ and R⁸ are as given in Table 1 for compounds 1-1 to 1-1230, are designated as compound numbers 19-1 to 19-1230 respectively.
1230 compounds of formula (I), wherein X¹ and X² are sulfur, X³ is oxygen, R¹ and R² are methyl, R⁴ is chloro, R¹⁶ and R¹⁷ are methyl, and Y is N, and the values of m, B, D, R³ and R⁸ are as given in Table 1 for compounds 1-1 to 1-1230, are designated as compound numbers 20-1 to 20-1230 respectively.
1230 compounds of formula (I), wherein X¹ is sulfur, X² and X³ are oxygen, R¹ and R² are methyl, R⁴ is CN, R¹⁶ and R¹⁷ are methyl, and Y is N, and the values of m, B, D, R³ and R⁸ are as given in Table 1 for compounds 1-1 to 1-1230, are designated as compound numbers 21-1 to 21-1230 respectively.
1230 compounds of formula (I), wherein X¹ and X² are sulfur, X³ is oxygen, R¹ and R² are methyl, R⁴ is CN, R¹⁶ and R¹⁷ are methyl, and Y is C—H, and the values of m, B, D, R³ and R⁸ are as given in Table 1 for compounds 1-1 to 1-1230, are designated as compound numbers 22-1 to 22-1230 respectively.
1230 compounds of formula (I), wherein X¹ is sulfur, X² and X³ are oxygen, R¹ and R² are methyl, R⁴ is bromo, R¹⁶ and R¹⁷ are methyl, and Y is N, and the values of m, B, D, R³ and R⁸ are as given in Table 1 for compounds 1-1 to 1-1230, are designated as compound numbers 23-1 to 23-1230 respectively.
1230 compounds of formula (I), wherein X¹ and X² are sulfur, X³ is oxygen, R¹ and R² are methyl, R⁴ is bromo, R¹⁶ and R¹⁷ are methyl, and Y is C—H, and the values of m, B, D, R³ and R⁸ are as given in Table 1 for compounds 1-1 to 1-1230, are designated as compound numbers 24-1 to 24-1230 respectively.

Compounds of the invention may be prepared by techniques known to the person skilled in the art of organic chemistry. General methods for the production of compounds of formula (I) are described below. Unless otherwise stated in the text, the substituents m, X¹, X², X³, Y, B, D, R¹, R², R³, R⁴, R⁵, R⁶, R⁷, R⁸, R⁹, R¹⁰, R¹¹, R¹², R¹³, R¹⁴, R¹⁵, R¹⁶ and R¹⁷ are as defined hereinbefore. The starting materials used for the preparation of the compounds of the invention may be purchased from usual commercial suppliers or may be prepared by known methods. The starting materials as well as the intermediates may be purified before use in the next step by state of the art methodologies such as chromatography, crystallization, distillation and filtration.

Compounds of formula (I) may be prepared from compounds of formula (A) and compounds of formula (B) as shown in reaction scheme 1.

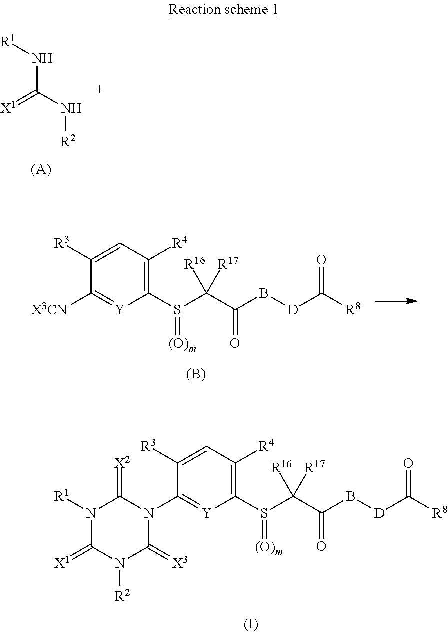

For example, a mixture of a compound of formula (A) and a compound of formula (B) may be treated with a base, such as triethylamine, and a carbonyl transfer reagent, such as phosgene or carbonyl diimidazole, in a suitable solvent such as toluene. Ureas or thioureas of formula (A) are available or may be prepared by methods well known in the literature. Compounds of formula (B) may be prepared from anilines of formula (C) as shown in reaction scheme 2.

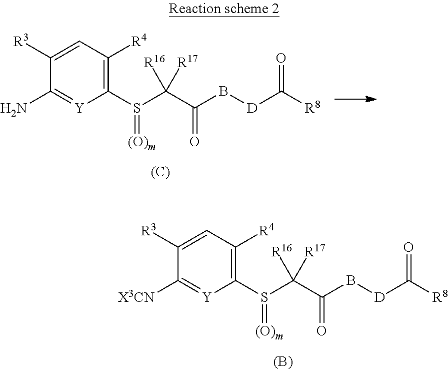

For example, a compound of formula (C) may be treated with a carbonyl or thiocarbonyl transfer reagent, such as diphosgene, triphosgene or thiophosgene, in a suitable solvent, such as toluene. Anilines of formula (C) may be prepared from nitro compounds of formula (D) as shown in reaction scheme 3.

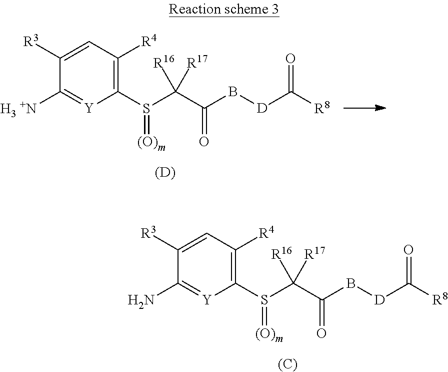

For example, a compound of formula (D) can be treated with a reducing agent, such as iron and ammonium chloride, in a suitable solvent, such as a mixture of water and ethanol. Nitro compounds of formula (D) in which m=0 may be prepared from thiols of formula (E) and compounds of formula (F), in which LG represents a leaving group, for example a halogen atom, such as a chlorine atom, as shown in reaction scheme 4.

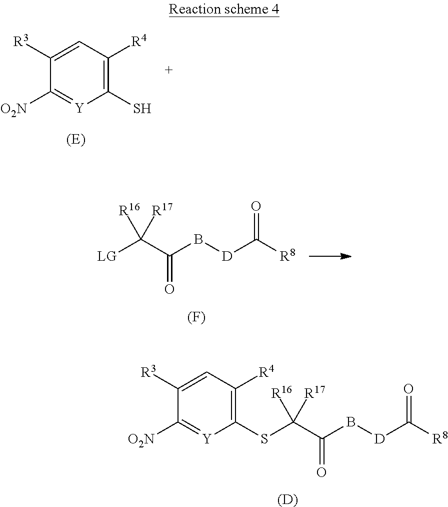

For example, a nitro compound of formula (E) may be treated with a compound of formula (F) in the presence of a base, such as triethylamine, in a suitable solvent such as acetonitrile. Nitro compounds of formula (E) are available or may be prepared by methods well known in the literature. Compounds of formula (F) may be prepared from alcohols or amines of formula (G) and acids of formula (H) as shown in reaction scheme 5.

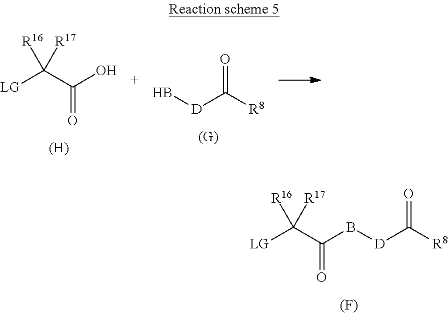

For example, an acid of formula (H) may be treated with an activating agent such as oxalyl chloride in a suitable solvent, such as dichloromethane and dimethylformamide, and the resulting intermediate then treated with an alcohol or amine of formula (G) in the presence of a base, such as triethylamine, in a suitable solvent such as dichloromethane. Alcohols and amines of formula (G) and acids of formula (H) are available or may be prepared by methods well known in the literature. Alternatively compounds of formula (I-A), which are compounds of formula (I) in which m=0, may be prepared from compounds of formula (F) and thiols of formula (J) as shown in reaction scheme 6.

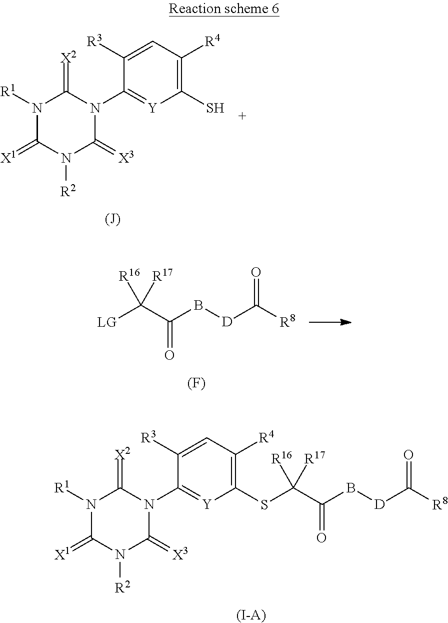

For example, a thiol of formula (J) may be treated with a compound of formula (F) in the presence of a base, such as triethylamine, in a suitable solvent such as acetonitrile. Thiols of formula (J) may be prepared from sulphonyl chlorides of formula (K) as shown in reaction scheme 7.

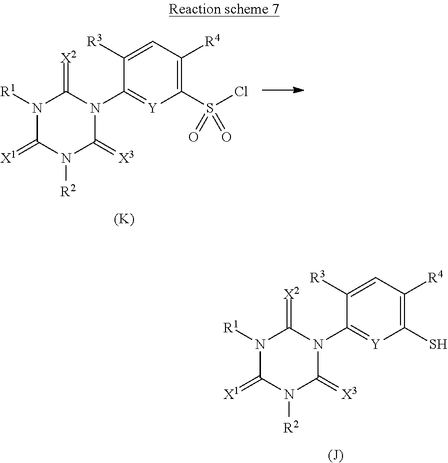

For example, a sulphonyl chloride of formula (K) may be treated with a reducing agent, such as tin dichloride, in a suitable solvent, such as a mixture of water and acetic acid. Sulphonyl chlorides of formula (K) can be prepared from compounds of formula (L) as shown in reaction scheme 8.

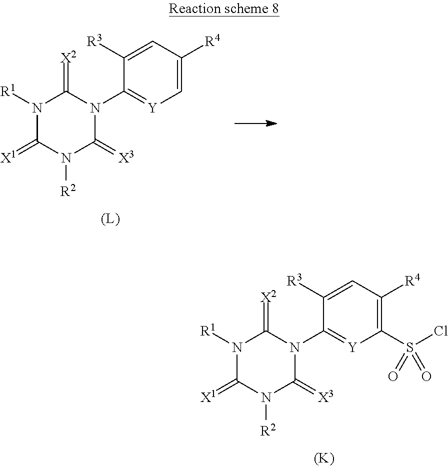

For example, a compound of formula (L) may be treated with a sulphonylating agent, such as chlorosulphonic acid. Compounds of formula (L) can be prepared from compounds of formula (A) and compounds of formula (M) as shown in reaction scheme 9.

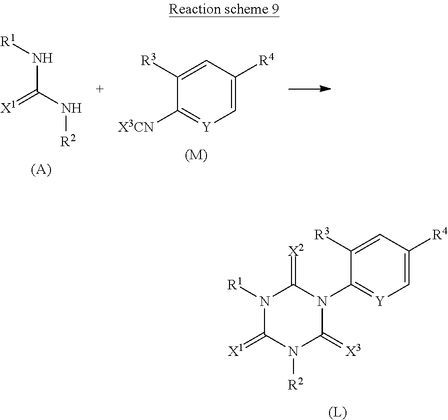

For example, a mixture of a compound of formula (A) and a compound of formula (M) may be treated with a base, such as triethylamine, and a carbonyl transfer reagent, such as phosgene or carbonyl diimidazole, in a suitable solvent such as toluene. Compounds of formula (M) may be prepared from anilines of formula (N) as shown in reaction scheme 10.

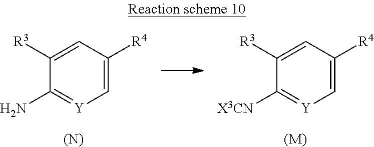

For example, a compound of formula (N) may be treated with a carbonyl orthiocarbonyl transfer reagent, such as diphosgene, triphosgene or thiophosgene, in a suitable solvent, such as toluene. Anilines of formula (N) are available or may be prepared by methods well known in the literature. Compounds of formula (I) may also be prepared from acids of formula (P) and alcohols or amines of formula (G) as shown in reaction scheme 11.

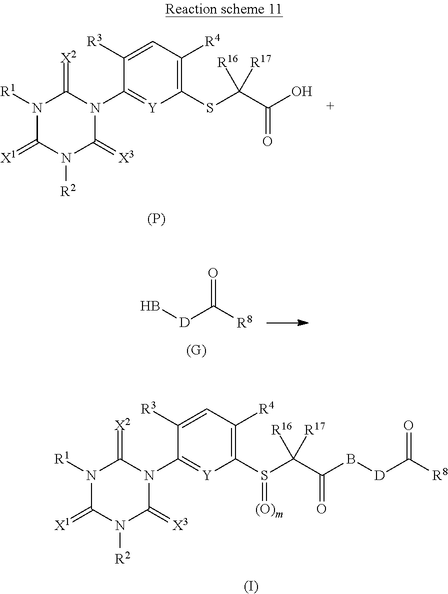

For example, an acid of formula (P) may be treated with an activating agent such as oxalyl chloride in a suitable solvent, such as dichloromethane and dimethylformamide, and the resulting intermediate then treated with an alcohol or amine of formula (G) in the presence of a base, such as triethylamine, in a suitable solvent such as dichloromethane. Acids of formula (P) may be prepared from esters of formula (Q), in which R represents an alkyl or similar group, as shown in reaction scheme 12.

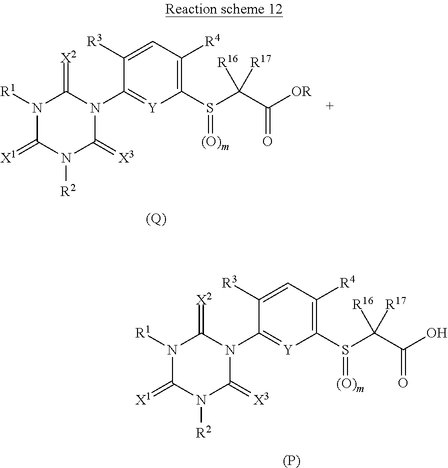

For example, a compound of formula (Q) may be treated with hydrochloric acid in a suitable solvent, such as dioxane. Esters of formula (Q) in which m=0 may be prepared from thiols of formula (J) and esters of formula (R), in which LG represents a leaving group, for example a halogen atom, such as a chlorine atom, and R represents an alkyl or similar group, as shown in reaction scheme 13.

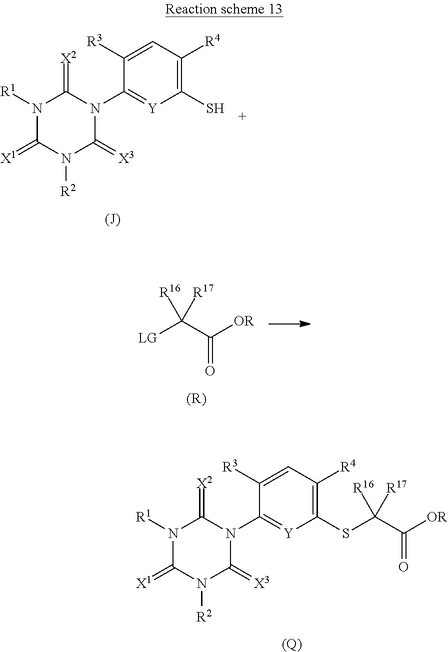

For example, a thiol of formula (J) may be treated with a compound of formula (R) in the presence of a base, such as triethylamine, in a suitable solvent such as acetonitrile. Esters of formula (R) are available or may be prepared by methods well known in the literature. Compounds of formula (I-B), which are compounds of formula (I) in which m=1 or 2, may be prepared from compounds of formula (I-A), which are compounds of formula (a) in which m=0, as shown in reaction scheme 14.

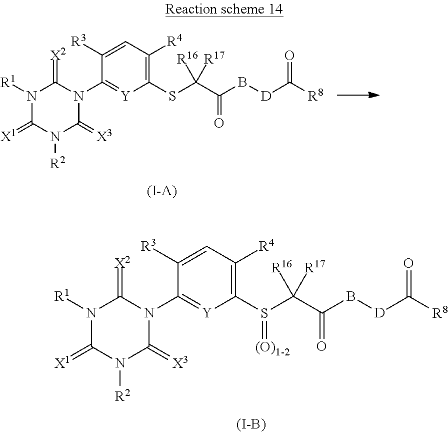

For example, a compound of formula (I-A) may be treated with an oxidising agent, for example oxone or metachloroperbenzoic acid, in a suitable solvent, such as acetonitrile or dichloromethane. Compounds of formula (I-C), which are compounds of formula (I) in which Rₐ is an OH group, may be prepared from compounds of formula (I-D) in which compounds of formula (I) in which R⁸ is OR⁹, as shown in reaction scheme 15.

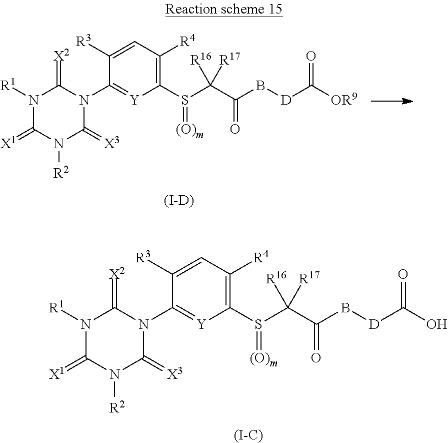

For example, a compound of formula (I-D) may be treated with hydrochloric acid in a suitable solvent, such as dioxane. Compounds of formula (I-E), which are compounds of formula (I) in which R⁸ is NR¹⁰R¹¹, may be prepared from compounds of formula (I-C) as shown in reaction scheme 16.

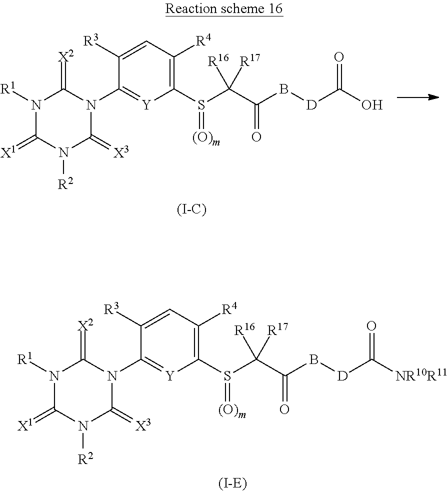

For example, a compound of formula (I-C) may be treated with a halogenating reagent, such as oxalyl chloride, in a suitable solvent, such as dichloromethane, to form an acyl halide which may be treated with a reagent HNR¹⁰R¹¹ in the presence of a base, such as triethylamine, in a suitable solvent, such as dichloromethane.

One skilled in the art will realise that it is often possible to alter the order in which the transformations described above are conducted, or to combine them in alternative ways to prepare a wide range of compounds of formula (I). Multiple steps may also be combined in a single reaction. All such variations are contemplated within the scope of the invention.

The skilled person will also be aware that some reagents will be incompatible with certain values or combinations of the substituents m, X¹, X², X³, Y, B, D, R¹, R², R³, R⁴, R⁵, R⁶, R⁷, R⁸, R⁹, R¹⁰, R¹¹, R¹², R¹³, R¹⁴, R¹⁵, R¹⁶ and R¹⁷ as defined herein, and any additional steps, such as protection and/or deprotection steps, which are necessary to achieve the desired transformation will be clear to the skilled person.

The compounds according to the invention can be used as herbicidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). For water-soluble compounds, soluble liquids, water-soluble concentrates or water soluble granules are preferred. Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The herbicidal compositions generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha.

Preferred formulations can have the following compositions (weight %):

| | |
|---|---|
| Emulsifiable concentrates: | |
| active ingredient: | 1 to 95%, preferably 60 to 90% |
| surface-active agent: | 1 to 30%, preferably 5 to 20% |
| liquid carrier: | 1 to 80%, preferably 1 to 35% |
| Dusts: | |
| active ingredient: | 0.1 to 10%, preferably 0.1 to 5% |
| solid carrier: | 99.9 to 90%, preferably 99.9 to 99% |
| Suspension concentrates: | |
| active ingredient: | 5 to 75%, preferably 10 to 50% |
| water: | 94 to 24%, preferably 88 to 30% |
| surface-active agent: | 1 to 40%, preferably 2 to 30% |
| Wettable powders: | |
| active ingredient: | 0.5 to 90%, preferably 1 to 80% |
| surface-active agent: | 0.5 to 20%, preferably 1 to 15% |
| solid carrier: | 5 to 95%, preferably 15 to 90% |
| Granules: | |
| active ingredient: | 0.1 to 30%, preferably 0.1 to 15% |
| solid carrier: | 99.5 to 70%, preferably 97 to 85% |

The composition of the present may further comprise at least one additional pesticide. For example, the compounds according to the invention can also be used in combination with other herbicides or plant growth regulators. In a preferred embodiment the additional pesticide is a herbicide and/or herbicide safener.

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, fomesafen flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, [(E)-[2-(trifluoromethyl)phenyl]methyleneamino]2,6-bis[(4,6-dimethoxypyrimidin-2-yl)oxy]benzoate, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H-indol-6-yl)pyridine-2-carboxylate), 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl-2-[[3-[[3-chloro-5-fluoro-6-[3-methyl-2,6-dioxo-4-(trifluoromethyl)pyrimidin-1-yl]-2-pyridyl]oxy]acetate, 6-chloro-4-(2,7-dimethyl-1-naphthyl)-5-hydroxy-2-methyl-pyridazin-3-one, tetrahydrofuran-2-ylmethyl (2R)-2-[(4-amino-3,5-dichloro-6-fluoro-2-pyridyl)oxy]propanoate, (2R)-2-[(4-amino-3,5-dichloro-6-fluoro-2-pyridyl)oxy]propanoic acid, tetrahydrofuran-2-ylmethyl 2-[(4-amino-3,5-dichloro-6-fluoro-2-pyridyl)oxy]propanoate, 2-[(4-amino-3,5-dichloro-6-fluoro-2-pyridyl)oxy]propanoic acid, 2-fluoro-N-(5-methyl-1,3,4-oxadiazol-2-yl)-3-[(R)-propylsulfinyl]-4-(trifluoromethyl)benzamide, 2-fluoro-N-(5-methyl-1,3,4-oxadiazol-2-yl)-3-propylsulfinyl-4-(trifluoromethyl)benzamide, (2-fluorophenyl)methyl 6-amino-5-chloro-2-(4-chloro-2-fluoro-3-methoxy-phenyl)pyrimidine-4-carboxylate, 6-amino-5-chloro-2-(4-chloro-2-fluoro-3-methoxy-phenyl)pyrimidine-4-carboxylic acid, 3-(3-chlorophenyl)-6-(5-hydroxy-1,3-dimethyl-pyrazole-4-carbonyl)-1,5-dimethyl-quinazoline-2,4-dione and [4-[3-(3-chlorophenyl)-1,5-dimethyl-2,4-dioxo-quinazoline-6-carbonyl]-2,5-dimethyl-pyrazol-3-yl]N,N-diethylcarbamate, methyl 2-[(E)-[2-chloro-4-fluoro-5-[3-methyl-2,6-dioxo-4-(trifluoromethyl)pyrimidin-1-yl]phenyl]methyleneamino]oxypropanoate and methyl (2R)-2-[(E)-[2-chloro-4-fluoro-5-[3-methyl-2,6-dioxo-4-(trifluoromethyl)pyrimidin-1-yl]phenyl]methyleneamino]oxypropanoate.

The mixing partners of the compound of formula (I) may also be in the form of esters or salts, as mentioned e.g. in The Pesticide Manual, Fourteenth Edition, British Crop Protection Council, 2006.

The compound of formula (I) can also be used in mixtures with other agrochemicals such as fungicides, nematicides or insecticides, examples of which are given in The Pesticide Manual.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

Compounds of formula (I) of the present invention may also be combined with herbicide safeners. Preferred combinations benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of formula (I) with cyprosulfamide, isoxadifen (including isoxadifen-ethyl), cloquintocet (including cloquintocet-mexyl) and/or N-(2-methoxybenzoyl)-4-[(methyl-aminocarbonyl)amino]benzenesulfonamide.

The safeners of the compound of formula (I) may also be in the form of esters or salts, as mentioned e.g. in The Pesticide Manual, 14^{th} Edition (BCPC), 2006. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

The compounds of formula (I) of this invention are useful as herbicides. The present invention therefore further comprises a method for controlling unwanted plants comprising applying to the said plants or a locus comprising them, an effective amount of a compound of the invention or a herbicidal composition containing said compound. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of formula (I) may vary within wide limits and depend on the nature of the soil, the method of application (pre-emergence; post-emergence; application to the seed furrow; no tillage application etc.), the crop plant, the weed(s) to be controlled, the prevailing climatic conditions, and other factors governed by the method of application, the time of application and the target crop. The compounds of formula (I) according to the invention are generally applied at a rate of from 10 to 2000 g/ha, especially from 50 to 1000 g/ha. A preferred range is 10-200 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

Compounds of formula (I) and compositions of the invention can typically be used to control a wide variety of monocotyledonous and dicotyledonous weed species. Examples of monocotyledonous species that can typically be controlled include Alopecurus myosuroides, Avena fatua, Brachiaria plantaginea, Bromus tectorum, Cyperus esculentus, Digitaria sanguinalis, Echinochloa crus-galli, Lolium perenne, Lolium multiflorum, Panicum miliaceum, Poa annua, Setaria viridis, Setaria faberi and Sorghum bicolor. Examples of dicotyledonous species that can be controlled include Abutilon theophrasti, Amaranthus retroflexus, Bidens pilosa, Chenopodium album, Euphorbia heterophylla, Galium aparine, Ipomoea hederacea, Kochia scoparia, Polygonum convolvulus, Sida spinosa, Sinapis arvensis, Solanum nigrum, Stellaria media, Veronica persica and Xanthium strumarium.

Unwanted plants are to be understood as also including those weeds that have been rendered tolerant to herbicides or classes of herbicides (e.g. ALS-, GS-, EPSPS-, PPO-, ACCase- and HPPD-inhibitors) by evolution, by conventional methods of breeding or by genetic engineering. Examples include Amaranthus palmeri that has evolved resistance to glyphosate and/or acetolactate synthase (ALS) inhibiting herbicides.

The compounds of the present invention can be used in methods of controlling unwanted plants or weeds which are resistant to protoporphyrinogen oxidase (PPO) inhibitors. For example, Amaranthus palmeri and Amaranthus tuberculatus populations have evolved as PPO-resistant weeds e.g. due to amino acid substitutions in PPX2L such as those occurring at amino acids R128 (also referred to as R98) and G399, or a codon (glycine) deletion in PPX2L at codon 210 (Δ210), the codon numbering being based on NCBI reference DQ386114. The compounds of the present invention can be used in methods of controlling Amaranthus palmeri and/or Amaranthus tuberculatus with mutations or deletions at the previously mentioned codons or equivalents, and it would be obvious to try the compounds to control unwanted plants or weeds with other mutations conferring tolerance or resistance to PPO inhibitors that may arise.

The compounds of formula (I) are also useful for pre-harvest desiccation in crops, for example, but not limited to, potatoes, soybean, sunflowers and cotton. Pre-harvest desiccation is used to desiccate crop foliage without significant damage to the crop itself to aid harvesting.

Compounds/compositions of the invention are particularly useful in non-selective burn-down applications, and as such may also be used to control volunteer or escape crop plants.

Various aspects and embodiments of the present invention will now be illustrated in more detail by way of example. It will be appreciated that modification of detail may be made without departing from the scope of the invention.

### EXAMPLES

The Examples which follow serve to illustrate, but do not limit, the invention.

### Synthesis Examples

### Example 1 Preparation of (3-methoxy-3-oxo-propyl) 2-[2-chloro-5-(3,5-dimethyl-2,6-dioxo-4-thioxo-1,3,5-triazinan-1-yl)-4-fluoro-phenyl]sulfanylpropanoate (Compound 9-245)

### Step 1: Synthesis of 2-chloro-4-fluoro-5-nitro-benzoic acid

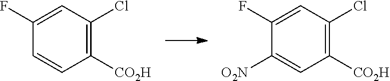 [SMILES: O=C(O)c1ccc(F)cc1Cl ; O=C(O)c1cc([N+](=O)[O-])c(F)cc1Cl]

Fuming nitric acid (2.8 ml, 43 mmol) was added to a stirred solution of 2-chloro-4-fluoro-benzoic acid (5.0 g, 29 mmol) in concentrated sulphuric acid (20 ml) at 0° C. The mixture was stirred for 20 minutes then poured carefully into iced water. The resulting mixture was filtered and the solid washed with acetonitrile to provide 2-chloro-4-fluoro-5-nitro-benzoic acid (6.4 g). ¹H NMR (400 MHz, CDCl₃) δ 8.6 (d, 1H), 7.05 (d, 1H), 5.9 (br s, 1H) ppm.

### Step 2: Synthesis of ethyl 2-chloro-4-fluoro-5-nitro-benzoate

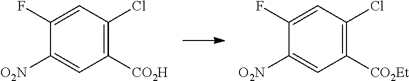 [SMILES: O=C(O)c1cc([N+](=O)[O-])c(F)cc1Cl ; CCOC(=O)c1cc([N+](=O)[O-])c(F)cc1Cl]

Oxalyl chloride (0.88 ml, 9.9 mmol) was added dropwise to a solution of 2-chloro-4-fluoro-5-nitro-benzoic acid (1.5 g, 6.6 mmol) in dichloromethane (15 ml). The solution was stirred for 15 minutes then dimethylformamide (3 drops) was added and stirring continued until the evolution of gas ceased. Ethanol (4 ml, 67 mmol) was added and the resulting solution stirred for 17 hours, then the solvent evaporated under reduced pressure to provide ethyl 2-chloro-4-fluoro-5-nitro-benzoate (1.7 g) as a solid. ¹H NMR (400 MHz, CDCl₃) δ 8.7 (d, 1H), 7.5 (d, 1H), 4.45 (q, 2H), 1.45 (t, 3H) ppm.

### Step 3: Synthesis of ethyl 5-amino-2-chloro-4-fluoro-benzoate

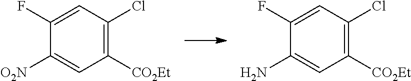 [SMILES: CCOC(=O)c1cc([N+](=O)[O-])c(F)cc1Cl ; CCOC(=O)c1cc(N)c(F)cc1Cl]

Tin dichloride hydrate (4.7 g, 24 mmol) was added to a stirred solution of ethyl 2-chloro-4-fluoro-5-nitro-benzoate (2.1 g, 8.1 mmol) in ethyl acetate (21 ml) and the mixture then heated at reflux for 1.5 hours, allowed to cool and the solvent evaporated under reduce pressure. The residue was purified by column chromatography to provide ethyl 5-amino-2-chloro-4-fluoro-benzoate (1.2 g). ¹H NMR (400 MHz, CDCl₃) δ 7.3 (d, 1H), 7.1 (d, 1H), 4.35 (q, 2H), 3.35 (br s, 2H), 1.4 (t, 3H) ppm.

### Step 4: Synthesis of ethyl 2-chloro-4-fluoro-5-isocyanato-benzoate

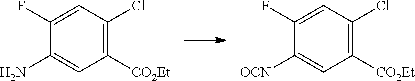

Diphosgene (0.8 ml, 6.6 mmol) was added to a stirred solution of ethyl 5-amino-2-chloro-4-fluoro-benzoate (1.2 g, 5.5 mmol) in dry toluene (24 ml). The resulting mixture was heated at reflux for 2 hours, allowed to cool and concentrated under reduced pressure to provide ethyl 2-chloro-4-fluoro-5-isocyanato-benzoate.

### Step 5: Synthesis of ethyl 2-chloro-5-(3,5-dimethyl-2,6-dioxo-4-thioxo-1,3,5-triazinan-1-yl)-4-fluoro-benzoate

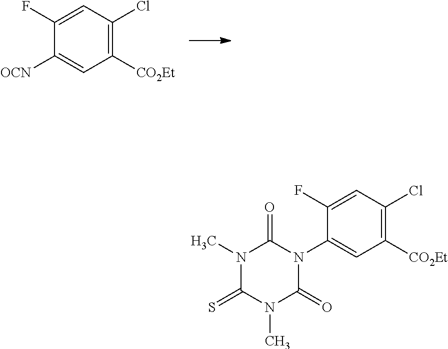

A solution of 1,3-dimethylthiourea (0.26 g, 2.5 mmol) and triethylamine (0.37 ml, 2.7 mmol) in toluene (10 ml) was added to ethyl 2-chloro-4-fluoro-5-isocyanato-benzoate (500 mg, 2.1 mmol) and the resulting solution heated to reflux. Carbonyl diimidazole (520 mg, 3.1 mmol) was added portionwise over 15 minutes and the resulting solution heated at reflux for a further 3 hours, then cooled and the solvent evaporated under reduced pressure. The residue was purified by column chromatography to provide ethyl 2-chloro-5-(3,5-dimethyl-2,6-dioxo-4-thioxo-1,3,5-triazinan-1-yl)-4-fluoro-benzoate (550 mg) as a gum. ¹H NMR (400 MHz, CDCl₃) δ 7.95 (d, 1H), 7.45 (d, 1H), 4.4 (q, 2H), 3.8 (s, 6H), 1.45 (t, 3H) ppm.

### Step 6: Synthesis of 2-chloro-5-(3,5-dimethyl-2,6-dioxo-4-thioxo-1,3,5-triazinan-1-yl)-4-fluoro-benzoic acid

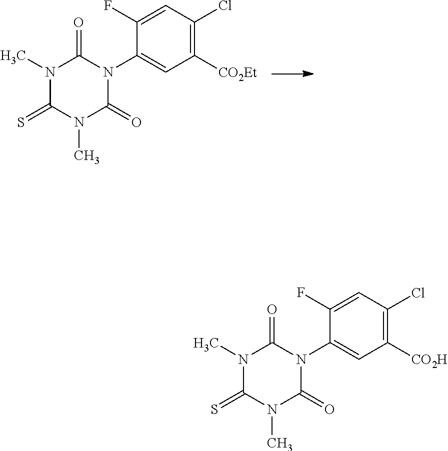 [SMILES: CCOC(=O)c1cc(-n2c(=O)n(C)c(=S)n(C)c2=O)c(F)cc1Cl ; Cn1c(=S)n(C)c(=O)n(-c2cc(C(=O)O)c(Cl)cc2F)c1=O]

Concentrated sulphuric acid (2.6 ml, 45 mmol) was added to a stirred solution of ethyl 2-chloro-5-(3,5-dimethyl-2,6-dioxo-4-thioxo-1,3,5-triazinan-1-yl)-4-fluoro-benzoate (1.7 g, 4.5 mmol) in glacial acetic acid (17 ml). The mixture was heated at 110° C. for 6 hours, allowed to cool to ambient temperature and carefully poured into iced water. The resulting mixture was filtered and the solid dried to provide 2-chloro-5-(3,5-dimethyl-2,6-dioxo-4-thioxo-1,3,5-triazinan-1-yl)-4-fluoro-benzoic acid (1.5 g). 1H NMR (400 MHz, DMSO-d₆) δ 8.15 (d, 1H), 7.85 (d, 1H), 3.6 (s, 6H), 3.35 (br s, 1H) ppm.

### Step 7: Synthesis of tert-butyl N-[2-chloro-5-(3,5-dimethyl-2,6-dioxo-4-thioxo-1,3,5-triazinan-1-yl)-4-fluoro-phenyl]carbamate

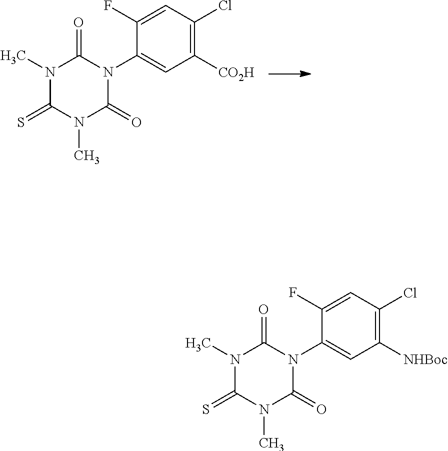 [SMILES: Cn1c(=S)n(C)c(=O)n(-c2cc(C(=O)O)c(Cl)cc2F)c1=O ; Cn1c(=S)n(C)c(=O)n(-c2cc(NC(=O)OC(C)(C)C)c(Cl)cc2F)c1=O]

A solution of dicyclohexylcarbodiimide (0.64 g, 3.0 mmol) in chlorobenzene (6 ml) was added dropwise to a stirred solution of 2-chloro-5-(3,5-dimethyl-2,6-dioxo-4-thioxo-1,3,5-triazinan-1-yl)-4-fluoro-benzoic acid (1.0 g, 2.9 mmol) and tert-butyl N-hydroxycarbamate (0.37 g, 2.7 mmol) in chlorobenzene (6 ml) at −15° C. The resulting mixture was stirred for 30 minutes, then palladium dichloride triphenylphosphine complex (0.1 g, 0.15 mmol) and caesium carbonate (1.9 g, 5.8 mmol) was added and the mixture heated at 85° C. for one hour. The mixture was cooled, extracted with ethyl acetate and the organic phase dried and evaporated to leave a residue which was purified by column chromatography to provide tert-butyl N-[2-chloro-5-(3,5-dimethyl-2,6-dioxo-4-thioxo-1,3,5-triazinan-1-yl)-4-fluoro-phenyl]carbamate (0.8 g). ¹H NMR (400 MHz, CDCl₃) δ 8.3 (d, 1H), 7.3 (d, 1H), 3.8 (s, 6H), 1.5 (s, 9H) ppm (NH not observed).

### Step 8: Synthesis of 3-(5-amino-4-chloro-2-fluoro-phenyl)-1,5-dimethyl-6-thioxo-1,3,5-triazinane-2,4-dione

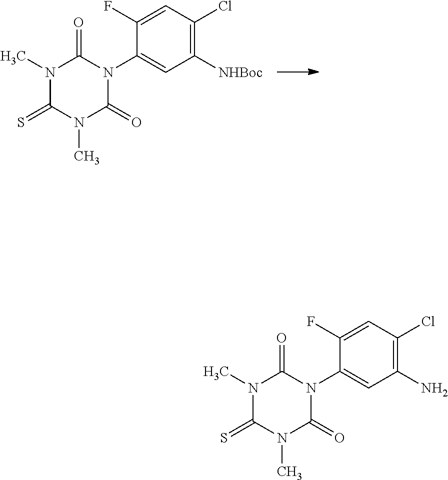 [SMILES: Cn1c(=S)n(C)c(=O)n(-c2cc(NC(=O)OC(C)(C)C)c(Cl)cc2F)c1=O ; Cn1c(=S)n(C)c(=O)n(-c2cc(N)c(Cl)cc2F)c1=O]

A mixture of tert-butyl N-[2-chloro-5-(3,5-dimethyl-2,6-dioxo-4-thioxo-1,3,5-triazinan-1-yl)-4-fluoro-phenyl]carbamate (700 mg, 1.7 mmol) and trifluoroacetic acid (10.5 ml) was stirred at ambient temperature for 1 hour, then concentrated under reduced pressure. Saturated aqueous sodium bicarbonate (30 ml) was added and the mixture extracted with ethyl acetate (2×80 ml). The combined organic phases were dried over sodium sulphate, filtered and evaporated under reduced pressure to provide 3-(5-amino-4-chloro-2-fluoro-phenyl)-1,5-dimethyl-6-thioxo-1,3,5-triazinane-2,4-dione (500 mg) as a white solid. ¹H NMR (400 MHz, CDCl₃) δ 7.25 (d, 1H), 6.7 (d, 1H), 3.8 (s, 6H) ppm (NH₂ not observed).

### Step 9: Synthesis of 2-chloro-5-(3,5-dimethyl-2,6-dioxo-4-thioxo-1,3,5-triazinan-1-yl)-4-fluoro-benzenesulfonyl chloride

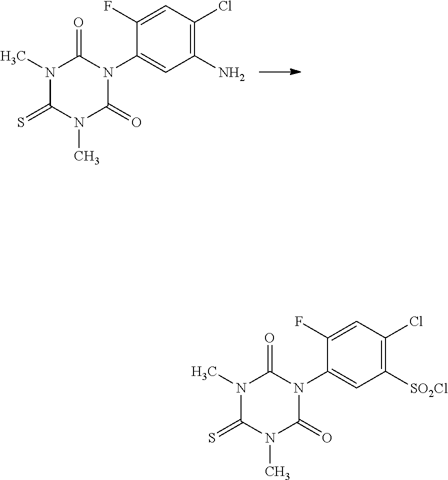 [SMILES: Cn1c(=S)n(C)c(=O)n(-c2cc(N)c(Cl)cc2F)c1=O ; Cn1c(=S)n(C)c(=O)n(-c2cc(S(=O)(=O)Cl)c(Cl)cc2F)c1=O]

A solution of sodium nitrite (0.24 g, 3.3 mmol) in water (12 ml) at 0° C. was added dropwise to a stirred mixture of 3-(5-amino-4-chloro-2-fluoro-phenyl)-1,5-dimethyl-6-thioxo-1,3,5-triazinane-2,4-dione (0.9 g, 2.8 mmol) and hydrochloric acid (24 ml) at −5° C. and the resulting mixture stirred at 0° C. for 15 minutes. The mixture was added dropwise to a stirred mixture of thionyl chloride (12 ml) and water (48 ml) to which copper (I) chloride (6 mg, 0.06 mmol) had been added at −5° C. The resulting mixture was stirred at 0° C. for 30 minutes and ambient temperature for 1 hour, then water (30 ml) and ethyl acetate (90 ml) added. The phases were separated and the aqueous extracted with ethyl acetate (90 ml). The combined organic phases were dried over sodium sulphate, filtered and evaporated under reduced pressure to provide 2-chloro-5-(3,5-dimethyl-2,6-dioxo-4-thioxo-1,3,5-triazinan-1-yl)-4-fluoro-benzenesulfonyl chloride (1 g) as a solid. ¹H NMR (400 MHz, CDCl₃) δ 8.2 (d, 1H), 7.6 (d, 1H), 3.8 (s, 6H) ppm.

### Step 10: Synthesis of 3-(4-chloro-2-fluoro-5-sulfanyl-phenyl)-1,5-dimethyl-6-thioxo-1,3,5-triazinane-2,4-dione

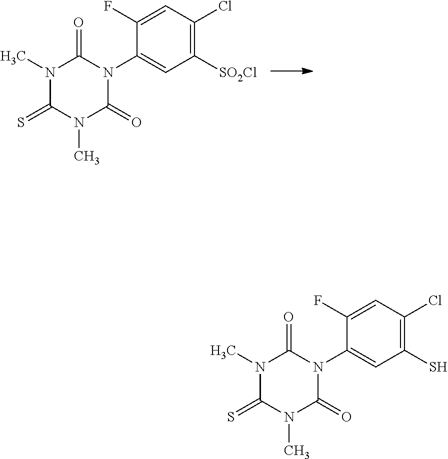 [SMILES: Cn1c(=S)n(C)c(=O)n(-c2cc(S(=O)(=O)Cl)c(Cl)cc2F)c1=O ; Cn1c(=S)n(C)c(=O)n(-c2cc(S)c(Cl)cc2F)c1=O]

Triphenylphosphine (0.94 g, 3.5 mmol) was added portionwise to a stirred solution of 2-chloro-5-(3,5-dimethyl-2,6-dioxo-4-thioxo-1,3,5-triazinan-1-yl)-4-fluoro-benzenesulfonyl chloride (0.5 g, 1.0 mmol) in tetrahydrofuran (6 ml). Water (1 ml) was added and the resulting mixture stirred at ambient temperature for 18 hours. Water (20 ml) and ethyl acetate (50 ml) were added and the pashes separated. The aqueous phase was extracted with ethyl acetate (50 ml) and the combined organic phases dried over sodium sulphate, filtered and evaporated under reduced pressure to leave a residue which was purified by column chromatography to provide 3-(4-chloro-2-fluoro-5-sulfanyl-phenyl)-1,5-dimethyl-6-thioxo-1,3,5-triazinane-2,4-dione (264 mg) as a solid. ¹H NMR (400 MHz, CDCl₃) δ 7.35 (m, 2H), 3.9 (s, 1H), 3.8 (s, 6H) ppm.

### Step 11: Synthesis of (3-methoxy-3-oxo-propyl) 2-[2-chloro-5-(3,5-dimethyl-2,6-dioxo-4-thioxo-1,3,5-triazinan-1-yl)-4-fluoro-phenyl]sulfanylpropanoate (Compound 9-245)

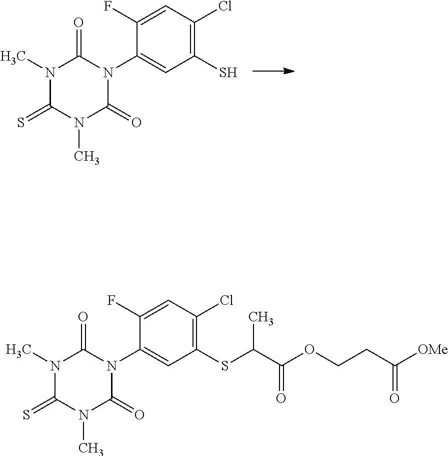 [SMILES: Cn1c(=S)n(C)c(=O)n(-c2cc(S)c(Cl)cc2F)c1=O ; COC(=O)CCOC(=O)C(C)Sc1cc(-n2c(=O)n(C)c(=S)n(C)c2=O)c(F)cc1Cl]

(3-Methoxy-3-oxo-propyl) 2-bromopropanoate (prepared as described in example 2; 70 mg, 0.29 mmol) was added to a stirred mixture of 3-(4-chloro-2-fluoro-5-sulfanyl-phenyl)-1,5-dimethyl-6-thioxo-1,3,5-triazinane-2,4-dione (90 mg, 0.26 mmol), caesium carbonate (88 mg, 0.27 mmol) and acetonitrile (1.8 ml) and the resulting mixture stirred at ambient temperature for 2 hours. Ethyl acetate (40 ml) was added and the resulting mixture washed with water (2×10 ml), dried over sodium sulphate, filtered and evaporated to leave a residue which was purified by column chromatography to provide (3-methoxy-3-oxo-propyl) 2-[2-chloro-5-(3,5-dimethyl-2,6-dioxo-4-thioxo-1,3,5-triazinan-1-yl)-4-fluoro-phenyl]sulfanylpropanoate (Compound 9-245) (85 mg) as an oil. ¹H NMR (400 MHz, CDCl₃) δ 7.55 (d, 1H), 7.4 (d, 1H), 4.3 (m, 2H), 3.9 (m, 1H), 3.75 (s, 6H), 3.65 (s, 3H), 2.6 (m, 2H), 1.55 (d, 3H) ppm.

### Example 2 Preparation of (3-methoxy-3-oxo-propyl) 2-bromopropanoate

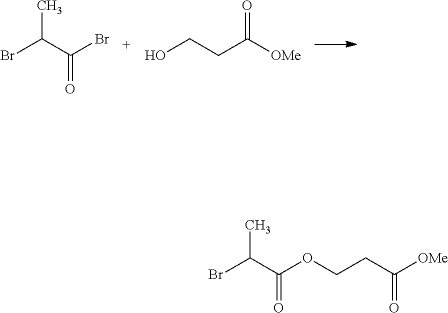

Triethylamine (1.3 ml, 9.1 mmol) and methyl 3-hydroxy propanoate (0.48 ml, 5.0 mmol) were added dropwise to a stirred solution of 2-bromopropanoyl bromide (0.49 ml, 4.5 mmol) in acetonitrile (15 ml) at 0° C. The resulting mixture was stirred at 0° C. for 1 hour, then ambient temperature for 2 hours. Water (20 ml) and ethyl acetate (60 ml) were added, the phases separated and the aqueous phase extracted with ethyl acetate (60 ml). The combined organic phases were dried over sodium sulphate, filtered and evaporated under reduced pressure to leave an oil, which was purified by column chromatography to provide (3-methoxy-3-oxo-propyl) 2-bromopropanoate (440 mg) as an oil. ¹H NMR (400 MHz, CDCl₃) δ 4.45 (m, 2H), 4.35 (m, 1H), 3.75 (s, 3H), 2.7 (t, 2H), 1.8 (d, 3H) ppm.

### Example 3 Preparation of methyl 3-[2-[2-chloro-5-(3,5-dimethyl-2,6-dioxo-4-thioxo-1,3,5-triazinan-1-yl)-4-fluoro-phenyl]sulfanylpropanoylamino]propanoate (Compound 9-272)

### Step 1: Synthesis of methyl 3-(2-bromopropanoylamino)propanoate

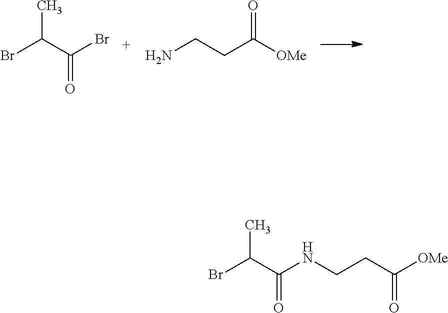

Triethylamine (2.6 ml, 9.1 mmol) and 2-bromopropanoyl bromide (0.97 ml, 9.1 mmol) were added to a stirred mixture of (3-methoxy-3-oxo-propyl)ammonium chloride (1.42 g, 10 mmol) in acetonitrile (30 ml) at 0° C. The resulting mixture was stirred at 0° C. for 1 hour, then ambient temperature for 2 hours. Water (20 ml) and ethyl acetate (60 ml) were added, the phases separated and the aqueous phase extracted with ethyl acetate (60 ml). The combined organic phases were dried over sodium sulphate, filtered and evaporated under reduced pressure to leave an oil, which was purified by column chromatography to provide methyl 3-(2-bromopropanoylamino)propanoate (560 mg) as a solid. ¹H NMR (400 MHz, CDCl₃) δ 6.95 (br s, 1H), 4.4 (q, 1H), 3.75 (s, 3H), 3.55 (m, 2H), 2.6 (m, 2H), 1.85 (d, 3H) ppm.

### Step 2: Synthesis of methyl 3-[2-[2-chloro-5-(3,5-dimethyl-2,6-dioxo-4-thioxo-1,3,5-triazinan-1-yl)-4-fluoro-phenyl]sulfanylpropanoylamino]propanoate (Compound 9-272)

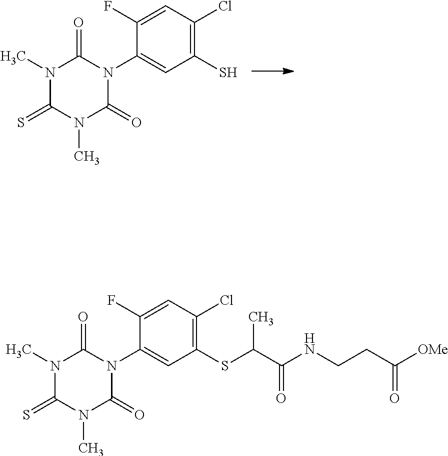 [SMILES: Cn1c(=S)n(C)c(=O)n(-c2cc(S)c(Cl)cc2F)c1=O ; COC(=O)CCNC(=O)C(C)Sc1cc(-n2c(=O)n(C)c(=S)n(C)c2=O)c(F)cc1Cl]

Methyl 3-(2-bromopropanoylamino)propanoate (78 mg, 0.32 mmol) was added to a stirred mixture of 3-(4-chloro-2-fluoro-5-sulfanyl-phenyl)-1,5-dimethyl-6-thioxo-1,3,5-triazinane-2,4-dione (prepared as described in Example 1, Step 10; 100 mg, 0.29 mmol), caesium carbonate (98 mg, 0.30 mmol) and acetonitrile (2 ml) and the resulting mixture stirred at ambient temperature for 2 hours. Ethyl acetate (50 ml) was added and the resulting mixture washed with water (2×20 ml), dried over sodium sulphate, filtered and evaporated to leave a residue which was purified by column chromatography to provide methyl 3-[2-[2-chloro-5-(3,5-dimethyl-2,6-dioxo-4-thioxo-1,3,5-triazinan-1-yl)-4-fluoro-phenyl]sulfanylpropanoylamino]propanoate (Compound 9-272) (36 mg) as a solid. ¹H NMR (400 MHz, CDCl₃) δ 7.35 (d, 1H), 7.25 (d, 1H), 7.0 (br m, 1H), 3.8 (m, 2H), 3.75 (s, 6H), 3.65 (s, 3H), 3.55 (m, 1H), 3.4 (m, 1H), 2.45 (m, 2H), 1.6 (d, 3H) ppm.

### Formulation Examples

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

Emulsifiable concentrate

| | |
|---|---|
| active ingredients | 10% |
| octylphenol polyethylene glycol ether (4-5 mol of ethylene oxide) | 3% |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### Biological Examples

### Pre-Emergence Biological Efficacy

Seeds of weeds and/or crops were sown in standard soil in pots (Amaranthus palmeri (AMAPA), Lolium perenne (LOLPE), Euphorbia heterophylla (EPHHL), Ipomoea hederacea (IPOHE), Setaria faberi (SETFA), Echinochloa crus-galli (ECHCG)). After cultivation for one day under controlled conditions in a glasshouse (at 24/19° C., day/night; 16 hours light), the plants were sprayed with an aqueous spray solution derived from the formulation of the technical active ingredient in a small amount of acetone and a special solvent and emulsifier mixture referred to as IF50 (11.12% Emulsogen EL360 TM+44.44% N-methylpyrrolidone+44.44% Dowanol DPM glycol ether), to create a 50 g/l solution which was then diluted using 0.2% Genapol XO80 as diluent to give the desired final dose of test compound.

The test plants were then grown under controlled conditions in the glasshouse (at 24/18° C., day/night; 15 hours light; 50% humidity) and watered twice daily. After 13 days the test was evaluated (100=total damage to plant; 0=no damage to plant). The results are shown in Table 2 below.

TABLE 2

| | Rate | Species | | | | | |
|---|---|---|---|---|---|---|---|
| Compound | (g/ha) | AMAPA | LOLPE | EPHHL | IPOHE | SETFA | ECHCG |
| 9-245 | 250 | 100 | 80 | 100 | 90 | 100 | 80 |
| 9-272 | 250 | 100 | 90 | 100 | 100 | 100 | 80 |

### Post-Emergence Biological Efficacy

Seeds of weeds and/or crops were sown in standard soil in pots (Amaranthus palmeri (AMAPA), Chenopodium album (CHEAL), Euphorbia heterophylla (EPHHL), Ipomoea hederacea (IPOHE), Eleusine indica (ELEIN), Lolium perenne (LOLPE), Digitaria sanguinalis (DIGSA), Setaria faberi (SETFA), Echinochloa crus-galli (ECHCG)). After cultivation for 14 days under controlled conditions in a glasshouse (at 24/19° C., day/night; 16 hours light), the plants were sprayed with an aqueous spray solution derived from the formulation of the technical active ingredient in a small amount of acetone and a special solvent and emulsifier mixture referred to as IF50 (11.12% Emulsogen EL360 TM+44.44% N-methylpyrrolidone+44.44% Dowanol DPM glycol ether), to create a 50 g/l solution which was then diluted using 0.2% Genapol XO80 as diluent to give the desired final dose of test compound.

The test plants were then grown under controlled conditions in the glasshouse (at 24/18° C., day/night; 15 hours light; 50% humidity) and watered twice daily. After 13 days the test was evaluated (100=total damage to plant; 0=no damage to plant). The results are shown in Table 3 below.

TABLE 3

| | Rate | Species | | | | | | | | |
|---|---|---|---|---|---|---|---|---|---|---|
| Compound | (g/ha) | AMAPA | CHEAL | EPHHL | IPOHE | ELEIN | LOLPE | DIGSA | SETFA | ECHCG |
| 9-245 | 250 | 100 | 100 | 90 | 100 | 80 | 100 | 30 | 40 | 70 |
| 9-272 | 250 | 80 | 90 | 90 | 100 | 80 | 100 | 70 | 90 | 40 |

1. A compound of formula (I) or an agronomically acceptable salt thereof: 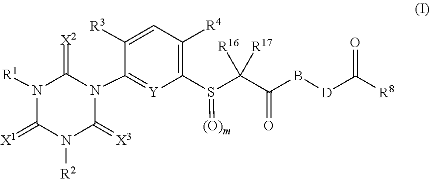
wherein
each X¹, X² and X³ is independently selected from oxygen and sulfur;
Y is C—H or nitrogen;
B is O, S, or NR⁵;
D is (CR⁶R⁷)ₙ;
m is an integer from 0 to 2;
n is an integer from 1 to 4;
R¹ is hydrogen or C₁-C₆alkyl;
R² is hydrogen, amino, C₁-C₆alkyl, C₃-C₆alkenyl, or C₃-C₆alkynyl;
R³ is hydrogen, halogen, C₁-C₄alkyl, C₁-C₄haloalkyl, C₁-C₄alkoxy, C₁-C₄haloalkoxy, C₁-C₄alkylthio, or C₁-C₄alkylsulfonyl;
R⁴ is hydrogen, halogen, cyano, nitro, aminocarbonyl, aminothiocarbonyl, C₁-C₄alkyl, C₁-C₄haloalkyl, C₁-C₄alkoxy, C₁-C₄haloalkoxy, or C₁-C₄alkylsulfonyl;
R⁵ is hydrogen, hydroxy, C₁-C₆alkyl, or C₁-C₄alkoxy;
Each R⁶ and R⁷ is independently selected from hydrogen, halogen, C₁-C₆alkyl, C₁-C₆haloalkyl, hydroxy, C₁-C₄alkoxy, C₁-C₄alkoxycarbonyl, or CH₂OR¹²; provided that R⁶ and R⁷ are not both hydroxy on the same carbon atom;
or two groups R⁶ and R⁷, on the same or different carbon atoms, together form a C₁-C₅alkylene chain, which contain 0, 1 or 2 oxygen atoms, substituted by 1-3 groups R⁵;
or two groups R⁶ and R⁷, on the same carbon atom, together with the carbon to which they are attached, form a C₂alkene;
R⁸ is OR⁹, SR⁹, or NR¹⁰R¹¹;
R⁹ is hydrogen, C₁-C₁₀alkyl, C₁-C₁₀haloalkyl, C₃-C₆alkenyl, C₃-C₆haloalkenyl, C₃-C₆alkynyl, C₁-C₄alkoxyC₁-C₆alkyl, C₁-C₄haloalkoxyC₁-C₆alkyl, C₆-C₁₀arylC₁-C₃alkyl, C₆-C₁₀arylC₁-C₃alkyl substituted by 1-4 groups R¹³, heteroarylC₁-C₃alkyl, or heteroarylC₁-C₃alkyl substituted by 1-3 groups R³;
R¹⁰ is hydrogen, C₁-C₆alkyl, or SO₂R¹⁴;
R¹¹ is hydrogen or C₁-C₆alkyl; or
R¹⁰ and R¹¹ together with the nitrogen to which they are attached form a 3- to 6-membered heterocyclyl ring, which optionally contains an oxygen atom;
R¹² is hydrogen, C₁-C₄alkyl, or C₁-C₄alkylcarbonyl;
each R¹³ is independently selected from halogen, C₁-C₄alkyl, C₁-C₄haloalkyl, C₁-C₄alkoxy, C₁-C₄haloalkoxy, cyano, and C₁-C₄alkylsulfonyl;
R¹⁴ is C₁-C₄alkyl, C₁-C₄haloalkyl, or C₁-C₄alkyl(C₁-C₄alkyl)amino;
each R¹⁵ is independently selected from hydrogen, halogen, C₁-C₄alkyl, and C₁-C₄haloalkyl;
R¹⁶ and R¹⁷ are independently selected from hydrogen, halogen, C₁-C₆alkyl, C₁-C₆haloalkyl, C₁-C₄alkoxy, and CH₂OR¹²;
or two groups R¹⁶ and R¹⁷, together form a C₂-C₅alkylene chain, which contains 0, 1 or 2 oxygen atoms, substituted by 1-3 groups R¹⁵;
or two groups R¹⁶ and R¹⁷ together with the carbon to which they are attached form a C₂alkene;
or a salt or N-oxide thereof.

2. A compound as claimed in claim 1, in which X¹ is sulfur.

3. A compound as claimed in claim 1, in which X² is oxygen.

4. A compound as claimed in claim 1, in which X³ is oxygen.

5. A compound as claimed in m claim 1, in which Y is C—H.

6. A compound as claimed in claim 1, in which B is O, NH, or NMe.

7. A compound as claimed in claim 1, in which n is an integer from 1 to 2.

8. A compound as claimed in claim 7, in which n is 2.

9. A compound as claimed in claim 1, in which R^{r} is hydrogen or C₁-C₄alkyl.

10. A compound as claimed in claim 1, in which R² is hydrogen, C₁-C₄alkyl, or C₃-C₄alkynyl.

11. A compound as claimed in claim 1, in which R³ is hydrogen, chloro, or fluoro.

12. A compound as claimed in claim 1, in which R⁴ is hydrogen, chloro, bromo, cyano, or aminothiocarbonyl.

13. A compound as claimed in claim 1, in which each R⁶ and R⁷ is independently selected from hydrogen, halogen, C₁-C₄alkyl, and C₁-C₄alkoxycarbonyl.

14. An agrochemical composition comprising a herbicidally effective amount of a compound of formula (I) as defined in claim 1 and an agrochemically-acceptable diluent or carrier.

15. A method of controlling or preventing undesirable plant growth, wherein a herbicidally effective amount of a compound of formula (I) as defined in claim 1 is applied to the plants, to parts thereof or to the locus thereof.
